(19) United States
(12) Patent Application Publication
(10) Pub. No.: US 2004/0171070 A1
(43) Pub. Date: Sep. 2, 2004

(54) Peptide analysis using a solid support

(76) Inventors: Ramagauri Bhikhabhai, Uppsala (SE); Maria Liminga, Uppsala (SE); Jean-Luc Maloisel, Uppsala (SE); Ronnie Palmgren, Uppsala (SE); Thomas W Keough, Cincinnati, OH (US); Amy M Keough, Cincinnati, OH (US); Robert S Youngquist, Mason, OH (US); Harold L Vaughn, Walton, KY (US); Ken E Yelm, Fairfield, OH (US)

Correspondence Address: AMERSHAM BIOSCIENCES, PATENT DEPARTMENT, 800 CENTENNIAL AVENUE, PISCATAWAY, NJ, 08855, US

(21) Appl. No.: 10/478,910

(22) PCT Filed: May 20, 2002
(86) PCT No.: PCT/US02/16247

(51) Int. Cl. G01N 33/53

(52) U.S. Cl. 435/7.1; 530/410

(57) ABSTRACT

The present invention relates to a method of identifying a polypeptide, which method comprises the steps of (a) derivatization of the N-terminus of the polypeptide, or the N-termini of one or more peptides of the polypeptide, with at least one acidic reagent which comprises a sulfonyl moiety coupled to an activated acid moiety to provide one or more peptide derivatives; analyzing at least one such derivative using a mass spectrometric technique to provide a fragmentation pattern, and (c) interpreting the fragmentation pattern obtained, wherein the peptide or polypeptide is immobilized to a solid support at least during step(a). Furthermore, the present invention also relates to a kit for identifying a polypeptide by a mass spectrometric technique.

### TECHNICAL FIELD

The present invention relates to an improved method of identifying a polypeptide, wherein an acidic reagent is used to derivatize peptides before analysis thereof using mass spectrometry. The invention also relates to a kit, which comprises reagent(s) suitable for use in the present method.

### BACKGROUND

The identification and sequencing of polypeptides has become of increased importance with the rapid development of the field of proteomics, wherein the expression products of novel genes are examined as to their function and composition.

Matrix-assisted laser desorption ionization (MALDI) mass spectrometry is a method developed for peptide and polypeptide sequencing. (For a reference to the principles of MALDI mass spectrometry, see e.g. Spengler et al., “Peptide Sequencing by Matrix-assisted Laser-desorption Mass Spectrometry”, Rapid Communications in Mass Spectrometry, Vol. 6, pp. 105-108 (1992).) MALDI mass spectrometry offers several advantages in the field of mass spectrometry. For example, it provides a higher sensitivity than the conventional electrospray triple quadrupole equipment. When used in combination with time-of-flight (TOF) mass analyzers, MALDI mass spectrometry is also applicable to higher mass peptides than can be analyzed with triple quadrupole equipment. MALDI mass spectrometry is also useful for analyzing complex mixtures with minimal sample purification. Electrospray ionization, on the other hand, is readily interfaced to powerful separation techniques including liquid chromatography (LC) and various forms of capillary electrophoresis (CE). Highly automated analyses are possible when using LC and CE as the sample purification and introduction devices.

However, current MALDI and, to a lesser extent, electrospray ionization mass spectrometric methods fail to adequately offer predictable tandem mass spectrometry fragmentation patterns. For example, multiple ion series (including a-ions, b-ions, and y-ions) are often observed, resulting in MALDI post-source decay spectra that are too complex for efficient interpretation and sequencing. Multiple ion series (b- and y-ions), plus internal fragments and both singly and multiply charged ions are formed from multiply charged precursor ions generated by electrospray ionization, and the resulting tandem mass spectra are often difficult to interpret de novo. Accordingly, problems with fragmentation have limited the ability to rapidly sequence polypeptides using mass spectrometry. As a result, mass spectrometry, and particularly MALDI mass spectrometry, has been of limited value in this area.

Several research groups have attempted to improve the utility of mass spectrometry in the field of polypeptide sequencing through the use of chemical derivatization techniques. Such techniques have been utilized to promote and direct fragmentation in the MSMS spectra of peptides with the goal of increasing sensitivity and decreasing the complexity of the resulting spectra. Most of these methods provide cationic derivatives. For example, derivatization with a quaternary ammonium group, and analysis using the static SIMS ionization method has been suggested. However, application of such techniques using MALDI mass spectrometry and electrospray ionization with low-energy collisional activation have not proven generally effective.

More recently, for the determination of an amino acid sequence, Keough et al (WO 00/43792, in the name of The Procter & Gamble Company) have suggested a derivatization of the N-terminus of a polypeptide with one or more acidic moieties having pKa values of less than 2 before analysis by mass spectrometry of the analyte, such as with MALDI mass spectrometry. The acidic moiety is preferably a sulfonic acid or a disulfonic acid derivative. The derivatives promote a charge-site-initiated cleavage of backbone amide bonds and they enable the selective detection of only a single series of fragment ions comprising the y-ions. However, the reaction according to Keough et al is generally performed under non-aqueous conditions due to the poor water stability of the reagents utilized therein. Accordingly, for a commercially useful determination of amino acid sequences by mass spectrometry, there is still a need for improved methods that fulfill the requirements especially for automated procedures.

### SUMMARY OF THE INVENTION

The object of the present invention is to provide a method of identification of a peptide or polypeptide using a mass spectrometric technique, which due to its robustness, sensitivity and easily interpreted fragmentation spectra is more suitable for automation than the prior art methods. This can be achieved by contacting acidic derivatization reagents with polypeptides immobilized to a solid support.

Thus, the present invention relates to a method of identifying a polypeptide, which method comprises the steps of:

(a) derivatization in an aqueous solution the N-terminus of the polypeptide, or the N-termini of one or more peptides of the polypeptide, with at least one acidic reagent comprising a sulfonyl or sulfonic acid moiety coupled to an activated acid moiety to provide one or more peptide derivatives, which reagent exhibits a half-life in aqueous solution of not less than 10 minutes, preferably not less than about 20 minutes and most preferably not less than about 30 minutes at room temperature;

(b) analyzing at least one such derivative using a mass spectrometric technique to provide a fragmentation pattern; and

(c) interpreting the fragmentation pattern obtained,

wherein the polypeptide is immobilized to a solid support at least during step (a).

The objects of the invention can more specifically be achieved as defined by the appended claims. Below, the present invention will be described in more detail with reference to specific embodiments and illustrative examples thereof.

### BRIEF DESCRIPTION OF THE DRAWINGS

FIG. 1 shows the reflectron spectrum of non-derivatized sample of horse myoglobin (15 fmol on MALDI target) as described in Example 2.

FIG. 2 shows the reflectron spectrum of derivatized sample (<15 fmol on the MALDI target) described in relation to FIG. 1.

FIG. 3 shows the PSD spectrum of m/z 1449.5, produced by derivatization of a lysine-terminated peptide (m/z 1271 as shown in FIG. 2).

FIG. 4 shows how the protein above was identified in PepFrag, by submitting the masses (−42 Da from the reaction) of the seven y-ions obtained.

FIG. 5 shows the fragmentation spectrum of an arginine-terminated peptide (m/z 1742.8).

FIG. 6 shows the eight y-ions obtained were used for protein identification in Pep-Frag.

FIG. 7 shows sulfonation of 500 femtomole of BSA tryptic peptides on solid phase as described in Example 3.

FIG. 8 shows sulfonation of 4.5 picomole of BSA tryptic peptides in solution as described in Example 3

FIG. 9A-D show NMR-spectra as discussed in Example 12 below.

FIG. 10A-B illustrate the stability of NHS-esters used according to the invention. More specifically, FIG. 10A shows the stability of 3-sulfopropionic acid NHS-ester in D₂O while FIG. 10B shows the stability of 2-sulfobenzoic acid NHS-ester in D₂O.

FIG. 11A-C show MALDI PSD spectra and comparative reactivity data of peptides sulfonated as described in Example 17.

FIG. 12 shows a reflectron spectrum, positive mode (showing average masses, after filtration, smoothing 5) of non-derivatized tryptic digest of 4VP-BSA obtained with the Ettan™MALDI-TOF.

FIG. 13 shows a reflectron spectrum (showing average masses, after filtration, smoothing 5) of derivatized tryptic digest of 4VP-BSA (Ettan™ MALDI-TOF).

FIG. 14 shows the PSD spectrum (positive mode) showing a complete y-ion series of peptide (I) from the derivatized tryptic digest of 4VP-BSA (FIG. 13) obtained with the Ettan™MALDI-TOF.

FIG. 15 shows the PSD spectrum (positive mode) of peptide (II) from the derivatized tryptic digest of 4VP-BSA (FIG. 13).

FIG. 16 shows the PSD spectrum (signals from 300 shots accumulated) of peptide (III) (FIG. 13), m/z1704, from the derivatized tryptic digest of 4VP-BSA.

FIG. 17 shows a first example of a reflectron spectrum (positive mode, 100 shots accumulated, showing average masses, after filtration, smoothing 5) of a non-derivatized protein digest from a Coomassie-stained 2-D gel obtained with the Ettan™ MALDI-TOF.

FIG. 18 shows a reflectron spectrum (positive mode, showing average masses, after filtration, smoothing 5) of the same 2-D sample as in FIG. 17 (remaining 95%), but after N-terminal derivatization with NHS-ester.

FIG. 19 shows a PSD spectrum (accumulated from 300 shots), of the derivatized peptide, m/z 1927.

FIG. 20 shows a second example of a reflectron spectrum (accumulated from 100 shots, showing average masses, after filtration, smoothing 5) of a non-derivatized tryptic digest of a protein spot from a Coomassie-stained 2D gel, obtained with Ettan™MALDI-TOF.

FIG. 21 shows a reflectron spectrum (positive mode showing average masses, after filtration, smoothing 5) of the same 2-D sample as in FIG. 19, but after ZipTip™ clean up and derivatization with NHS-ester in aqueous solution as described.

FIG. 22 shows a PSD spectrum (signal from 300 shots accumulated) of the derivatized peptide, m/z 1705 (see FIG. 12).

FIG. 23 shows sample-loaded ZipTips™ placed into a laboratory centrifuge for subsequent sulfonation in a multiplexed fashion.

FIG. 24 illustrates sample washing in the centrifuge following the sulfonation reaction.

FIG. 25 illustrates direct loading of the derivatized samples from the solid supports onto the MALDI sample stage.

FIG. 26 shows the MALDI mass spectra obtained following sulfonation of Fibrino-peptide A on solid support. Duplicate samples were sulfonated at three different peptide levels (10, 1 and 0.1 pmoles).

FIG. 27 shows the use of hydroxylamine hydrochloride for reversing unwanted ester side-products formed in the sulfonation reaction. The upper spectrum was obtained from ASHLGLAR sulfonated on solid support in the centrifuge. The lower spectrum was obtained from the same sulfonated peptide following treatment with hydroxylamine hydrochloride.

FIG. 28 demonstrates the sulfonation of a protein digest. The upper spectrum was obtained from the native protein digest. The lower spectrum was obtained following sulfonation of the digest.

### DEFINITIONS

In the present specification, the term “identifying” is not necessarily synonymous with determining the complete sequence, since it also includes partial sequence determination for identifying the polypeptide or characterizing it as similar to or different from a peptide derived from a known protein. Further, it also includes making a tentative identification based on the most probable of a small number of possibilities. Further, the term “ionization” as used herein refers to the process of creating or retaining on an analyte an electrical charge equal to plus or minus one or more electron units.

The term “aqueous environment” as used herein includes any water-based solution, suspension or any other form, which contains less than about 20% of organic solvents. As used herein, the term “electrospray ionization” refers to the process of producing ions from solution by electrostatically spraying the solution from a capillary electrode at high voltage with respect to a grounded counter electrode. The definition is intended to include both electrospray ionization and pneumatically assisted electrospray ionization, which is also referred to as ionspray. As used herein, the term “electrospray ionization” applies to all liquid flow rates and is intended to include microspray and nanospray experiments. Moreover, the definition is intended to apply to the analyses of peptides directly infused into the ion source without separation, and to the analysis of peptides or peptide mixtures that are separated prior to electrospray ionization. Suitable on-line separation methods include, but are not limited to, HPLC, capillary HPLC and capillary electrophoresis. Electrospray ionization experiments can be carried out with a variety of mass analyzers, including but not limited to, triple quadrupoles, ion traps, orthogonal-acceleration time-of-flight analyzers and Fourier Transform Ion Cyclotron Resonance instruments.

As used herein, the term “polypeptide” refers to a molecule having two or more amino acid residues.

As used herein, the term “wild-type” refers to a polypeptide produced by unmutated organisms.

As used herein, the term “variant” refers to a polypeptide having an amino acid sequence that differs from that of the wild-type polypeptide.

The term “water stable” as used herein refers reagents having a half-life in aqueous solution of not less than 10 minutes, preferably not less than about 20 minutes and most preferably not less than about 30 minutes at room temperature.

The term “activated acid” refers to an acid derivative, preferably a carboxylic acid derivative, which is capable of forming amide bonds in an aqueous environment.

The term “immobilized” as used herein to define how peptides and/or polypeptides are adsorbed to a solid support means that peptide and/or polypeptide binding is sufficiently strong to last during the reaction. For example, when the support is coated with C₁₈, a hydrophobic binding between the peptides and the support is strong enough to retain peptides through the reaction and cleanup steps.

As used herein, the following abbreviations are used:

| | |
|---|---|
| Tetrahydrofuran | THF |
| N-hydroxysuccinamide | NHS |
| Dichloromethane | DCM |
| N,N-diisopropylethylamine | DIEA |
| Trifluoroacetic acid | TFA |
| Deuterated water | D₂O |
| Hydrochloric acid | HCl |
| Thionyl chloride | SOCl₂ |
| Ethyl acetate | EtAc |
| Methanol | MeOH |
| Room Temperature and Pressure | RTP |
| Room Temperature | RT |
| Milli-Q purified water | MQ |
| O-(N-Succinimidyl)-N,N,N′,N′- | TSTU |
| tetramethyluronium BF₄ | |
| Acetonitrile | ACN |
| Deuterated chloroform | CDCl₃ |
| Thin layer chromatography | TLC |

### DETAILED DESCRIPTION OF THE INVENTION

A first aspect of the present invention is a method of identifying a polypeptide, which method comprises the steps of

(a) derivatization of the N-terminus of the polypeptide, or the N-termini of one or more peptides of the polypeptide, with a least one acidic reagent comprising a sulfonyl or sulfonic acid moiety coupled to an activated acid moiety to provide one or more peptide derivatives, which reagent exhibits a half-life in aqueous solution of not less than 10 minutes, preferably not less than about 20 minutes and most preferably not less than about 30 minutes at RT;

(b) analyzing at least one such derivative using a mass spectrometric technique to provide a fragmentation pattern; and

(c) interpreting the fragmentation pattern obtained,

wherein the peptide or polypeptide is immobilized to a solid support at least during step (a).

The solid support used according to the invention can be any suitable substrate capable of immobilizing peptides or polypeptides under the conditions defined herein. Thus, in one embodiment, the above-mentioned solid support is comprised of a silica-based medium derivatized with C₁₈. The solid support can e.g. be present on a plastic surface, such as the walls of microtiter wells, on a metal surface, such as a MALDI-slide, on the surface of a compact disc (Gyros A B, Uppsala, Sweden), or in composite structures, such as the commercially available ZipTip™ (Millipore Corporation, USA, see e.g. WO 98/37949). The high binding capacity of the present solid support results in a more efficient derivatization method. Also, the solid support is a convenient means to concentrate dilute peptide digests and to desalt e.g. prior to MALDI mapping, which greatly improves the signal/noise ratio. Other advantages of immobilizing polypeptides to a solid support is that it decreases reaction times, it reduces the number of sample manipulations required to guanidinate and/or sulfonate peptides and polypeptides and it increases the overall processing throughput. The spectra of protein digests that have been derivatized on solid supports often show increased numbers of tryptic peptides, improved protein sequence coverage and higher database search scores. In fact, the present inventors also have been able to show an improvement in sensitivity as high as five times that obtained using the corresponding chemistry but performed in solution instead of on a solid support. Materials such as ZipTip™ have not been used before as supports for peptide or polypeptide derivatization prior to mass spectrometry-based sequencing, but they been used simply to concentrate dilute solutions and to clean up the solutions by removing low-molecular weight contaminants such as alkali salts.

In an advantageous embodiment, the amount of ester side-products present after step (a) is reduced or eliminated by optionally adding a suitable chemical, such as hydroxylamine, mercaptoethanol, dithiothreitol or acetic hydrazide, that hydrolyzes unwanted ester groups. The derivatized peptide or polypeptide is washed to remove excess reagent prior to analysis. In the present context, the term “acidic” reagent means a reagent that comprises one or more moieties having pKa's of less than 2, preferably less than 0 and more preferably less than −2 when coupled to a peptide or polypeptide.

The present method is useful for sequencing polypeptides, such as wild-type, variant and/or synthetic polypeptides. The method is especially useful for identifying high molecular weight polypeptides for use e.g. in the biological and pharmaceutical field. More specifically, the present method can be used to facilitate biological studies requiring rapid determination of peptide or polypeptide sequences; to identify post-translational modifications in proteins and to identify amino acid modifications in variant proteins, such as those used in commercial laundry and cleansing products; to aid in the design of oligonucleotide probes for gene cloning; to rapidly characterize products formed in directed evolution studies; in combinatorial and peptide library identification; and in proteomics.

Thus, in step (b), the present invention utilizes a mass spectrometric technique for the analysis of the derivative(s), which technique can include matrix-assisted laser desorption ionization (MALDI) mass spectrometry or electrospry ionization. These ionization techniques can be carried out with a variety of mass analyzers, including but not limited to, triple quadrupoles, ion traps, reflector time-of-flight analysers, orthogonal-acceleration time-of-flight analyzers and Fourier Transform Ion Cyclotron Resonance instruments. The spectra obtained are routinely interpreted de novo in accordance with standard procedure. However, in the most preferred embodiment, in step (b), MALDI mass spectrometry is used. MALDI mass spectrometers are commercially available and described in the literature, see e.g. Kussmann M. and Roep-storff P., Spectroscopy 1998, 14:1-27.

Thus, as mentioned above, in the prior art sulfonic groups have been added to the N-termini of peptides to facilitate sequencing with MALDI mass spectrometry. Reagents suggested to this end include those exhibiting a low stability in water. (In this context, see e.g. T. Keough, R. S. Youngquist and M. P. Lacey, Proc.Natl. Acad. Sci. USA., 96, 7131 (1999); T. Keough, M. P. Lacey, A. M. Fieno, R. A. Grant, Y. Sun, M. D. Bauer and K. B. Begley, Electrophoresis, 66 2252 (1999); and T. Keough, M. P. Lacey and R. S. Youngquist, Rapid Commun. Mass Spectrom. 14,2348 (2000).) The present invention relates to a method wherein such acidic reagents are used, which method contrary to what has been suggested before is performed on polypeptides immobilized to a solid support. In the most advantageous embodiment, the present invention utilizes an acidic reagent comprised of a sulfonyl or sulfonic acid moiety coupled to an ester moiety, such as an NHS-ester. Such reagents will be discussed in more detail below.

Thus, in one embodiment, the present invention provides an improved one-step method wherein a water-stable reagent is used for the derivatization step preceding the actual mass spectrometry analyses. The advantages of working with a water-soluble and water stable reagent and avoiding organic solvents are obvious and include easier automation of the derivatization procedure because no dry down steps and solvent changes are required.

The fact that the present invention utilizes tryptic polypeptides immobilized to a solid support will also contribute to an enhanced suitability for automation. In an especially advantageous embodiment, both step (a) and a preceding guanidination step are performed on a solid support. This embodiment is advantageously performed simultaneously on a large number of samples, such as in the standard 96 well format in order to be easily adapted to available automation systems, such as ProSpot® (Amersham Biosciences AB, Uppsala, Sweden) or microfluidics sample preparation devices like compact disks (Gyros AB, Uppsala, Sweden). Such adaptation may include steps such as taking the solid support off pipettes, incubation etc. In this embodiment, the guanidination reaction and the sulfonation reaction are performed on the peptide or polypeptide contents of the same microtiter well, following immobilization on a ZipTip™. Accordingly, the samples need only be immobilized or bound once, which simplifies the procedure in total. Also, this embodiment has been shown to improve the sensitivity as much as 5 times as compared to the corresponding method in solution. As regards further differences between using peptides in solution and immobilized to a solid support for the present purpose, see Example 4 below, where a comparison of the sulfonation step is presented.

Furthermore, the present invention also relates to a method of protecting lysine residues by guanidination wherein the peptides and/or polypeptides are immobilized to a solid support.

In another embodiment, in order to reduce the duration of the sulfonation step and to provide an efficient derivatization procedure, the sulfonation reagent is centrifuged during step (a), which forces the liquids through the peptide or polypeptide-loaded ZipTips™, or any other solid phase used. This approach provides a mechanically simple means to move chemical reagents over immobilized peptides or polypeptides. The present inventors have unexpectedly shown that by using this embodiment, a near quantitative derivatization can be performed, see Example 3 below.

If the method according to this embodiment also includes a step for guanidination (as discussed in detail below), said reaction is conveniently performed during tryptic elution from a 2D gel, see e.g. Hale et al (Anal. Biochem. 28, (2000), 110-117). Guanidination during peptide extraction from the gel can be done robotically, and the tryptic peptides can subsequently be immobilized to a solid support and sulfonated as described above.

Accordingly, in an especially advantageous embodiment, the present method is a computer-assisted method, wherein suitable software is utilized in step (c). Thus, data analysis of mass-to-charge ratios obtained by the mass spectrometry is used for the interpretation of the fragmentation pattern obtained. Several software programs have been developed to compare mass spectra of the peptides obtained e.g. from MALDI-TOF experiments with theoretical spectra from proteins. The subject has been re-viewed by Kussmann and Roepstorff(Kussmann M. and Roepstorff P., Spectroscopy 1998, 14: 1-27).

An advantage of the kind of reagents used in the present method resides in the fact that they are easily stored in a crystalline form. Thus, the stability during storage and accordingly the shelf life of the reagents is greatly improved. Consequently, the present invention utilizes reagents that make possible a less costly handling and also simplifies the practical use thereof in many routine procedures.

The acidic reagent used in the present method may have a pKa of less than about 2, preferably less than about 0 and most preferably less than about −2 when coupled with a peptide or polypeptide. The skilled person in this field can measure pKa values of acidic moieties as covalently coupled to a polypeptide or peptide using standard methods well known in the art. For example, such methods may include titration or an electrochemical method. The activated acid moiety of the reagent can e.g. be an N-hydroxysuccinimide (NHS) ester, such as 3-sulfopropionic acid N-hydroxysuccinimide ester or 2-sulfobenzoic acid N-hydroxysuccinimide ester.

As the skilled in this field will realize, said reagent(s) can be used combined with any suitable buffer, as long as the buffer does not effectively compete with the analyte for the acidic reagent. In one embodiment, the buffer provides a pH within the range of about 8-12, such as 9-10 and in a specific embodiment about 9.4. One suitable buffer is 0.25 M NaHCO₃. Alternatively, they are simply used as dissolved in water, in which case the final solution pH will have to be adjusted, since the final solution pH must be basic for the reaction to occur. Furthermore, in the present method, it is to be understood that even though for practical reasons one single reagent is normally used, the invention also encompasses a method utilizing a mixture of two or more such reagents, each one of which being defined by comprising a sulfonyl or sulfonic acid moiety coupled to an NHS-ester moiety.

The preparation of the above mentioned exemplary reagents will be illustrated below in the experimental part of the present application. The activated acids used in the present method are prepared according to techniques well known to those ordinarily skilled in the art. The starting materials used in preparing the compounds of the invention are known, made by known methods, or are commercially available as a starting material.

It is recognized that the ordinarily skilled artisan in the art of organic chemistry can readily carry out standard manipulations of organic compounds without further direction. Examples of such manipulations are discussed in standard texts such as J. March, Advanced Organic Chemistry, John Wiley & Sons, 1992.

The ordinarily skilled artisan will readily appreciate that certain reactions are best carried out when other functionalities are masked or protected in the compound, thus increasing the yield of the reaction and/or avoiding any undesirable side reactions. Often, the ordinarily skilled artisan utilizes protecting groups to accomplish such increased yields or to avoid the undesired reactions. These reactions are found in the literature and are also well within the scope of the ordinarily skilled artisan. Examples of many such manipulations can be found in, for example, T. Greene, Protecting Groups in Organic Synthesis, John Wiley & Sons, 1981.

The compounds used in the present method may be prepared using a variety of procedures known to those ordinarily skilled in the art. Non-limiting general preparations include the following.

The activated acids used according to the invention can be prepared by activating the acid in a compound of the general structure below followed by reaction to generate a water stable reagent of the invention.

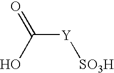

Where:

Y=a spacer which contains aliphatic and/or aromatic fragments and may optionally include additional sulfonic acids

Non-limiting examples of appropriate acids are e.g. 2-sulfoacetic acid, 3-sulfopropionic acid, 3-sulfobenzoic acid 4-sulfobenzoic acid, 2-bromo-5-sulfobenzoic acid and 2-sulfobenzoic acid. For a general reference to sulfonyl groups useful to this end, see e.g. WO 00/43792.

Those skilled in the art will realize that in addition to the protonated acids of these compounds, the salts including, but not limited to sodium and potassium will be useful for the synthesis of compounds of the invention. Most of the activated acids can be easily prepared with common methods of the art (Recent reviews and books for peptide synthesis and preparation of activated esters: a) Alberico, F.; Carpino, L. A., Coupling reagents and activation., Method. Enzymol., 1997, 289, 104-126. b) Bodansky, M.; Principles of Peptide Synthesis, 2^{nd} ed., Springer-Verlag: Berlin, 1993. c) Humphrey, J. M., Chamberlin, A. R., Chemical Synthesis of Natural Product Peptides: Coupling Methods for the Incorporation of Noncoded Amino Acids into Peptides. Chem. Rev., 1997, 97, 2243-2266. d) Handbook of Reagents for Organic Synthesis: Activating Agents and Protecting Groups, Pearson, A. J. and Roush, W. R., ed., John Wiley & Sons, 1999). Reactive derivatives of this structure include, for example, activated esters such as 1-hydroxybenzotriazole esters, mixed anhydrides of organic or inorganic acids such as hydrochloric acid and sulfonic acids, and symmetrical anhydrides of the acids of this structure. These activated materials may be directly useful as water-stable reagents of the invention. However; highly reactive materials such as acid chlorides may not be water stable as defined herein but can be further reacted with reagents such as N-hydroxysuccinamide to generate active acids that are water stable reagents of the invention.

Of the numerous active esters found in the literature, N-hydroxysuccinimide derived esters (Anderson, G. W.; Zimmerman, J. E.; Callahan, F. M.; J. Am. Chem. Soc., 1964, 86, 1839, For a review see Klausner, Y. S.; Bodansky, M. S., Synthesis, 1972, 453), ortho and para-nitrophenyl esters (Bodansky, M.; Funk, K. W., Fink, M. L.; J. Org. Chem., 1973, 38, 3565, Bodansky, M.; Du Vigneaud, V.; J. Am. Chem. Soc., 1959, 81, 5688), 2,4,5-trichlorophenyl esters (Pless, J.; Boissonnas, R. A., Helv. Chim. Acta; 1963, 46, 1609), pentachlorophenyl (Kovacs, J.; Kisfaludy, L., Ceprini, M. Q., J. Am. Chem. Soc., 1967, 89, 183) and pentafluorophenyl esters (Kisfaludy, L., Roberts, J. E., Johnson R. H., Mayers, G. L., Kovacs, J.; J. Org. Chem., 1970, 35, 3563) are of the most practical interest. Other acid activating moieties include, thio esters such as 2-pyridylthio esters (Lloyd, K.; Young, G. T.; J.Chem.Soc. (C), 1971, 2890), cyanomethyl esters (Schwyzer, R.; Iselin, B.; Feurer; M., Helv. Chim. Acta; 1955, 38, 69), N-acylimidazolides (Wieland, T.; Vogeler, K., Angew.Chem., 1961, 73, 435), acyl azide (Curtius, T., Ber.dtsch.Chem.Ges., 1902, 35, 3226 Fujii, N.; Yajima, H., J.Chem.Soc.Perkin Trans I, 1981, 789) or benzotriazol derived intermediate (Dormoy, J. R.; Castro, B., Tetrahedron, 1981, 37, 3699) are as well considered.

The use of these activated esters can as well be combined with selected acylation catalysts such as for example 4-dimethylaminopyridine (Hoefle, G.; Steglich, W.; Vorbrueggen, H., Angew. Chem., Int. Ed. Engl., 1978, 17, 569. Scriven, E. F. V., Chem.Soc.Rev., 1983,12, 129). The exact molecular structure of the reagent is not essential, as long as said sulfonyl or sulfonic acid moiety and the activated acid moiety are present and provided that its water stable nature and chemical reactivity with amines are retained. Further routine experimentation can subsequently be performed in order to identify e.g. an optimal pH for the reaction, or a specific activated acid, for which unwanted side reactions e.g. at hydroxyl groups are minimized.

The polypeptide, or peptides thereof, may be obtained by any means. For example, if necessary, the polypeptide of interest is isolated for analysis. Several procedures may be utilized for isolation including for example one-dimensional and two-dimensional electrophoresis. Alternatively, the polypeptides may have been synthesized through combinatorial chemistry methods well known in the art. In this instance, it is most preferable to synthesize a polypeptide having a basic or hydrophobic residue, preferably a basic (most preferably arginine or lysine), at or near the C-terminus of the resulting polypeptide.

Digestion may occur through any number of methods, including in-gel or on a membrane, preferably in-gel (see e.g. Shevchenko et al., “Mass Spectrometric Sequencing of Proteins from Silver-Stained Polyacrylamide Gels”, Analytical Chemistry, Vol. 68, pp. 850-858 (1996)). Thus, in an advantageous embodiment, the present method uses in—gel digests—. It is possible to digest the polypeptide either enzymatically or chemically, preferably enzymatically. It is most preferable to utilize a digestion procedure that yields a basic or hydrophobic residue, most preferably a basic, at or near the C-terminus of the resulting peptides.

A polypeptide may be digested enzymatically e.g. using trypsin, endoproteinase Lys C, endoproteinase Arg C, or chymotrypsin. Trypsin, endoproteinase Lys C or endo-proteinase Arg C are preferred, since the resulting peptides of the polypeptide will typically terminate at the C-terminus with an arginine or lysine residue (basic residue), with the exception of course of the C-terminus of the polypeptide. Other enzymes can be used, especially if basic residues occur at or near the C-terminus of the resulting peptides. For example, chymotrypsin, which typically cleaves at hydrophobic amino acid residues, may be used. Alternatively, chemical digestion can be used, such as by cyanogen bromide. (For a general reference to digestion methods, see e.g. U.S. Pat. No. 5,821,063.)

Thus, in a specific embodiment, the present method is used to identify a polypeptide or a protein, in which case a first step is included wherein said polypeptide or protein is digested, preferably enzymatically, to provide peptides. In a preferred embodiment, the enzyme is trypsin.

In an especially advantageous embodiment, the present method also includes a step of protecting specific residues before the derivatization step. For example, in a case where a polypeptide or protein is digested by trypsin, Lys residues may be protected in order to avoid e.g. undesired sulfonation reactions. An example of such a protection procedure by guanidination will be described in detail below in the experimental section (see Example 8). Guanidination is advantageously used, since it is capable of selectively protecting Lys side chains without having any adverse effect on peptide recovery in subsequent steps such as mapping experiments. Furthermore, guanidinated lysine residues in intact proteins are susceptible to trypsin digestion, so lysine-containing peptides can be used for a quantitative analysis. For example, a set of control proteins can be guanidinated with a reagent like O-methylisourea hydrogen-sulfate consisting of natural abundance isotopes. A treatment set of proteins can be guanidinated with the same reagent enriched in heavy isotopes e.g. O-methylisourea hydrogensulfate containing ¹³C and/or ¹⁵N. The protein mixtures can be combined and separated prior to tryptic digestion. Interesting proteins are identified with MALDI mapping and sequencing, and they are quantitated by comparing abundance ratios of isotopically labeled and unlabeled lysine-containing peptides.

The present method is preferably used with polypeptides from protein digests. Polypeptides can be used which preferably includes less than about 50 amino acid residues, more preferably less than about forty residues, even more preferably less than about thirty residues, still more preferably less than about twenty residues and most preferably less than about ten amino acid residues.

A second aspect of the present invention is the chemical compound 3-sulfopropionic acid N-hydroxysuccinimide ester as such, which is especially useful as a reagent for peptide derivatization on a solid support, as discussed above.

A third aspect of the present invention is the chemical compound 2-sulfobenzoic acid N-hydroxysuccinimide ester as such, which is also useful as a reagent for peptide derivatization on a solid support, as discussed above.

A fourth aspect of the invention is a kit for identifying a polypeptide, which kit contains an acidic reagent in a suitable container. The acidic reagent comprises a sulfonyl or sulfonic acid moiety coupled to an activated acid moiety, and is preferebly be present in the kit in the solid state. In one embodiment, the reagent is pre-weighed, and in an alternative embodiment, it is present as a bulk reagent. Such kit may also contain a buffer providing a pH within the range of 8-11. For reasons of stability, the buffer solution will be added by the end-users just prior to use. A kit according to the invention can also comprise a model peptide. The kit can also be accompanied by written instructions, e.g. in the form of a booklet, as to the use thereof.

Thus, in one embodiment, the present kit contains the necessary devices and means for performing a method of identifying a peptide or polypeptide according to the invention. A specific embodiment is a kit which comprises one or more of the novel reagents according to the invention and further means necessary for use with matrix-assisted laser desorption ionization time of flight (MALDI-TOF) mass spectrometry. An alternative embodiment is a kit, which comprises one or more of the novel reagents according to the invention and further means necessary for use with electrospray ionization mass spectrometry (ESI-MS). In a specific embodiment, the present kit also comprises hydroxylamine hydrochloride in a compartment separate from that of the reagent, which is useful to add to the reaction after finalized derivatization in order to reverse any unwanted ester side-products that have been formed by reaction with internal amino acids having side-chain hydroxyl groups.

A fifth aspect of the present invention is the use of an acidic reagent comprising a sulfonyl or sulfonic acid moiety coupled to an ester moiety, such as an N-hydroxy-succinimide (NHS) ester, e.g. a 3-sulfopropionic acid N-hydroxysuccinimide ester or a 2-sulfobenzoic acid N-hydroxysuccinimide ester, as a derivatization reagent in a mass spectrometric technique wherein the peptides are immobilized to a solid support during derivatization. More specifically, the present invention relates to the use of the above-described reagent in a method according to the invention.

### DETAILED DESCRIPTION OF THE DRAWINGS

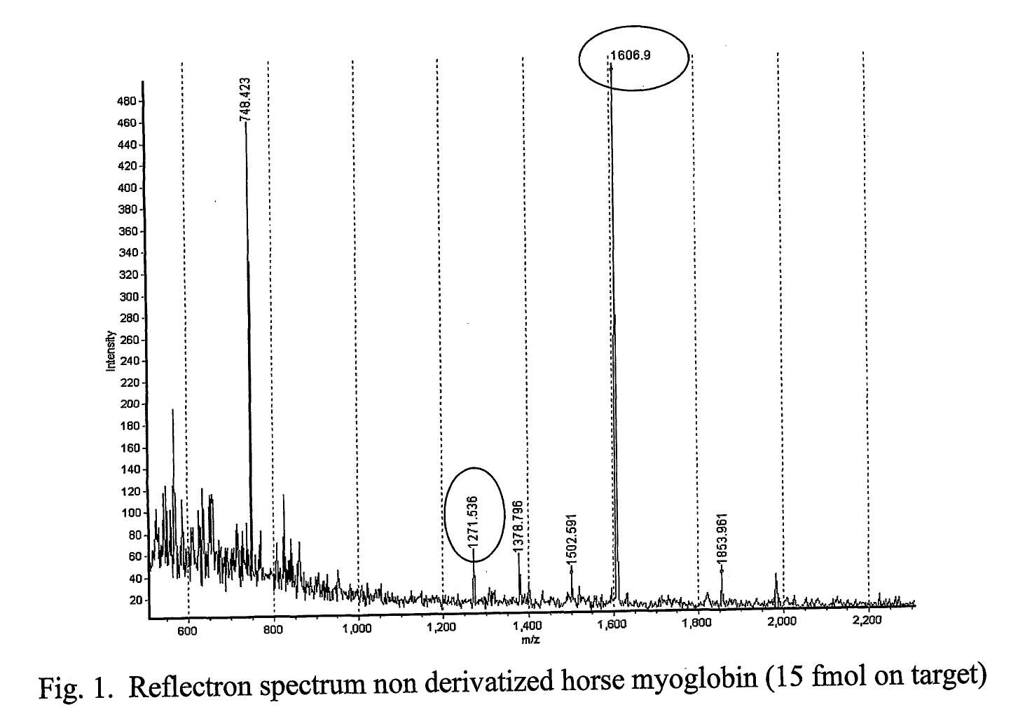

FIG. 1 shows the reflectron spectrum of non-derivatized sample of horse myoglobin (15 fmol on MALDI target) as described in Example 2 below.

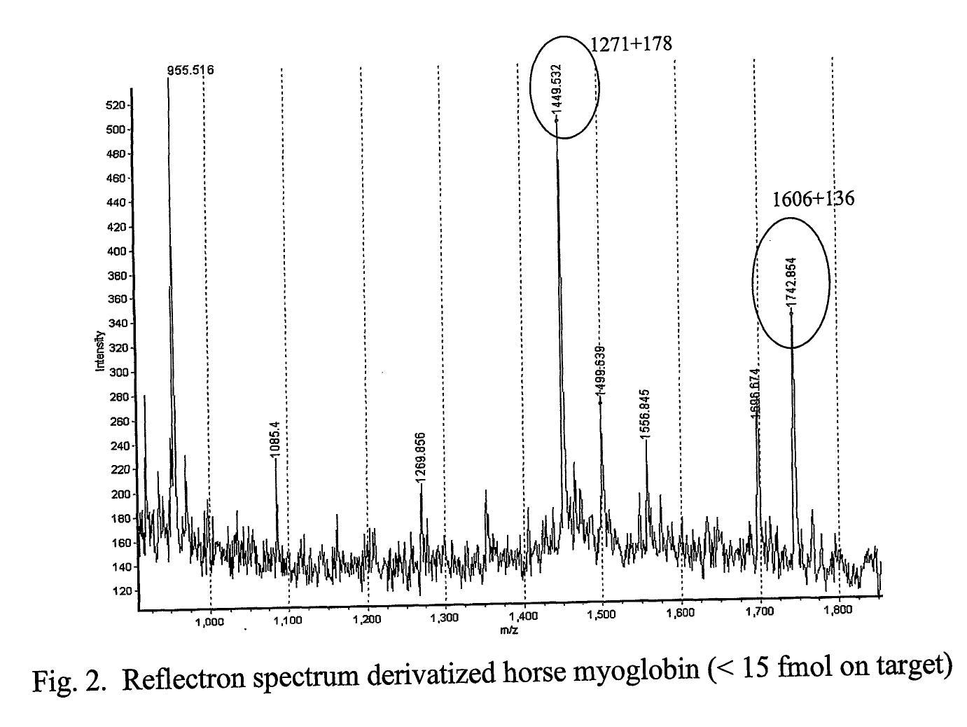

FIG. 2 shows the reflectron spectrum of derivatized sample (<15fmol on the MALDI target) described in relation to FIG. 1. Due to the efficient guanidination of the lysines on solid support, and the improved response of guanidinated peptides, the signals for the lysine-terminated peptides were dramatically increased in the reflectron spectrum of derivatized sample compared to the analysis of the non-derivatized sample. Two derivatized peptides were used for PSD analysis (one lysine terminated peptide m/z, 1449.5 and one arginine terminated peptide m/z, 1742.8).

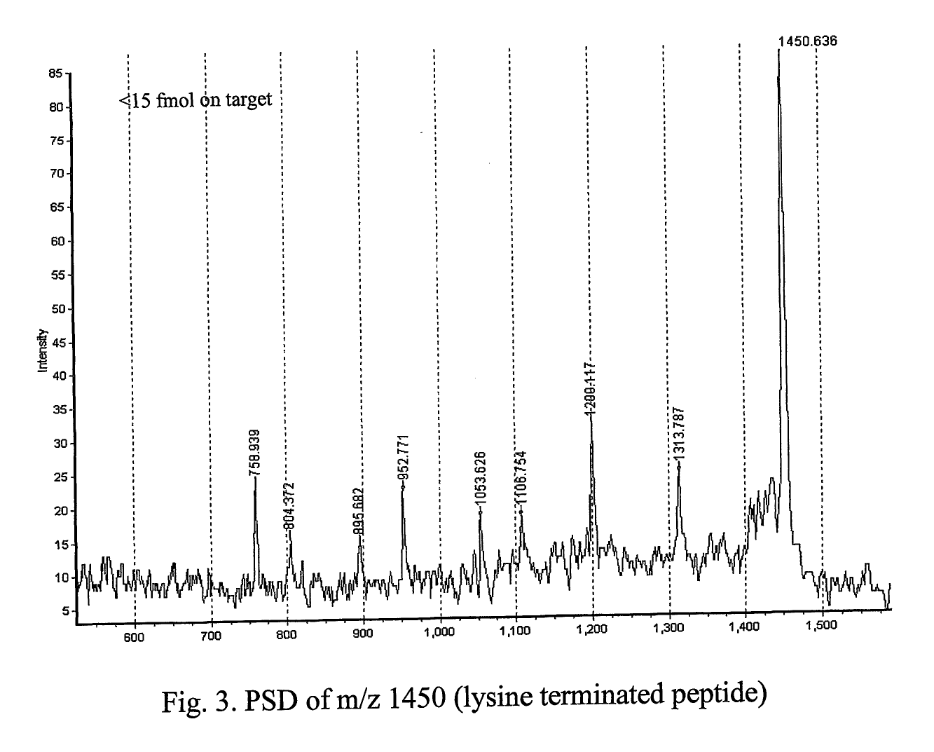

FIG. 3 shows the PSD spectrum of m/z 1449.5.

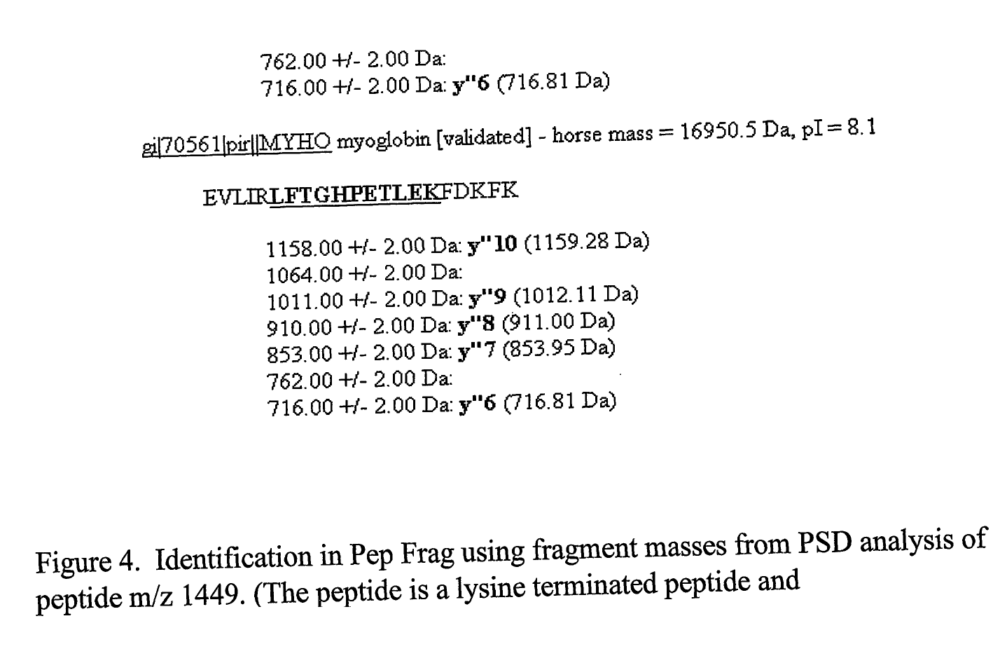

FIG. 4 shows how the protein above was identified in PepFrag, by submitting the masses of the seven observed y-ions (−42 Da mass increment resulting from the guanidination reaction).

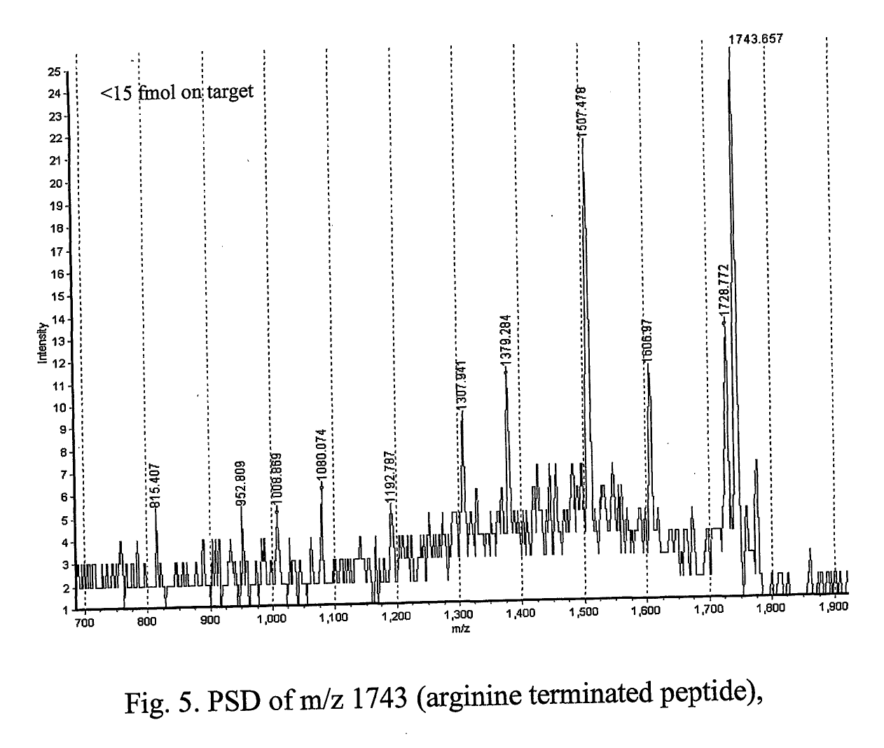

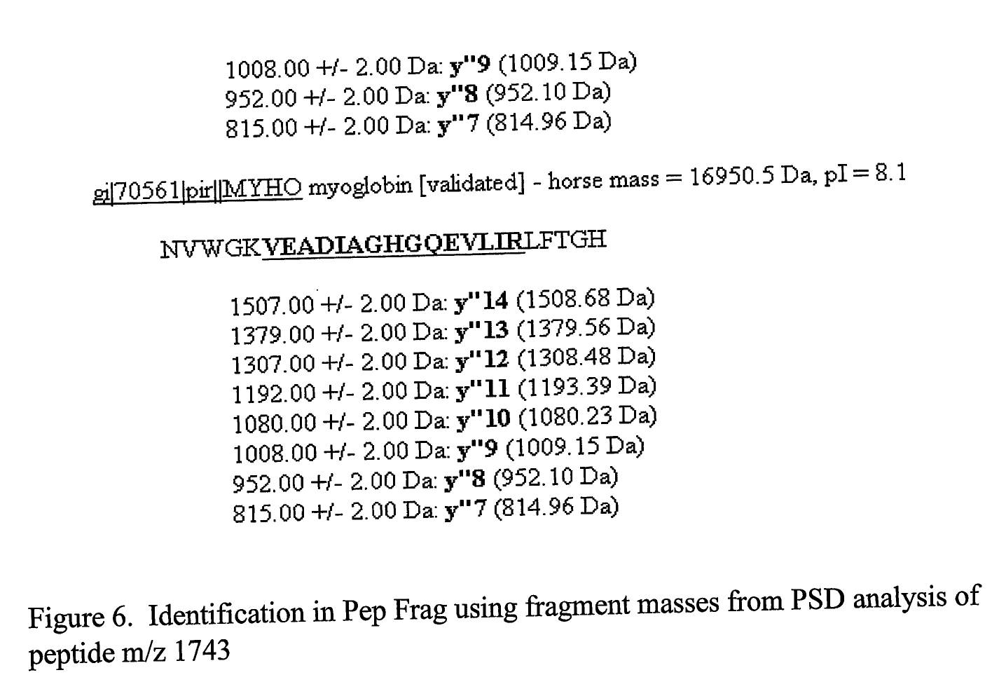

FIG. 5 shows the fragmentation spectrum of an arginine-terminated peptide (m/z 1742.8).

FIG. 6 shows the eight y-ions obtained were used for protein identification in Pep-Frag.

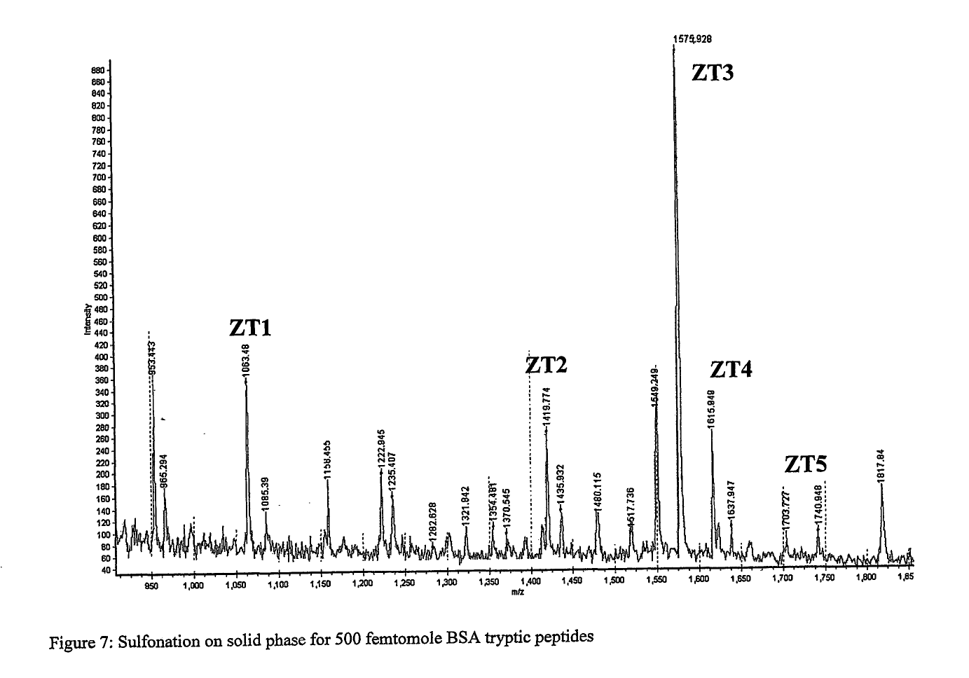

FIG. 7 shows sulfonation of 500 femtomoles of BSA tryptic peptides on solid phase as described in Example 3.

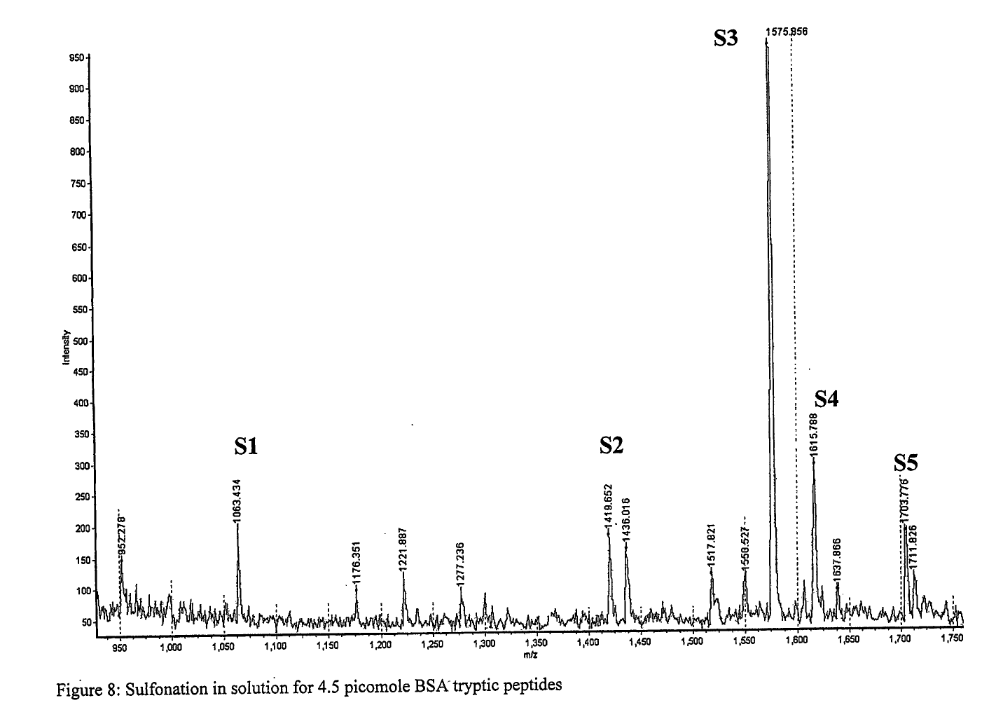

FIG. 8 shows sulfonation of 4.5 picomoles of BSA tryptic peptides in solution as described in Example 3

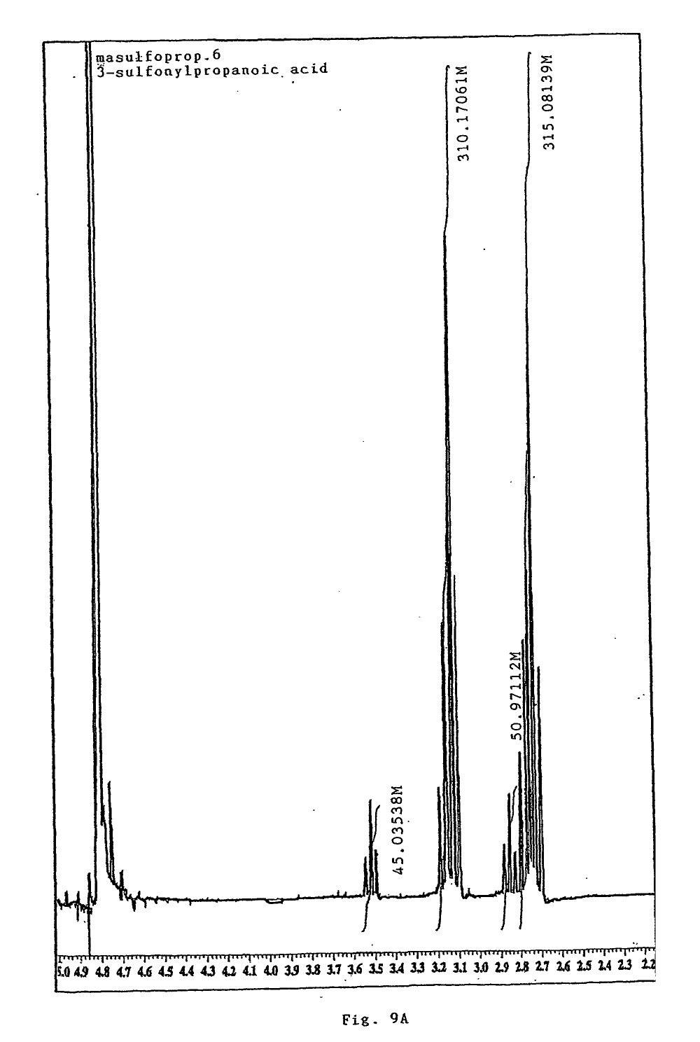

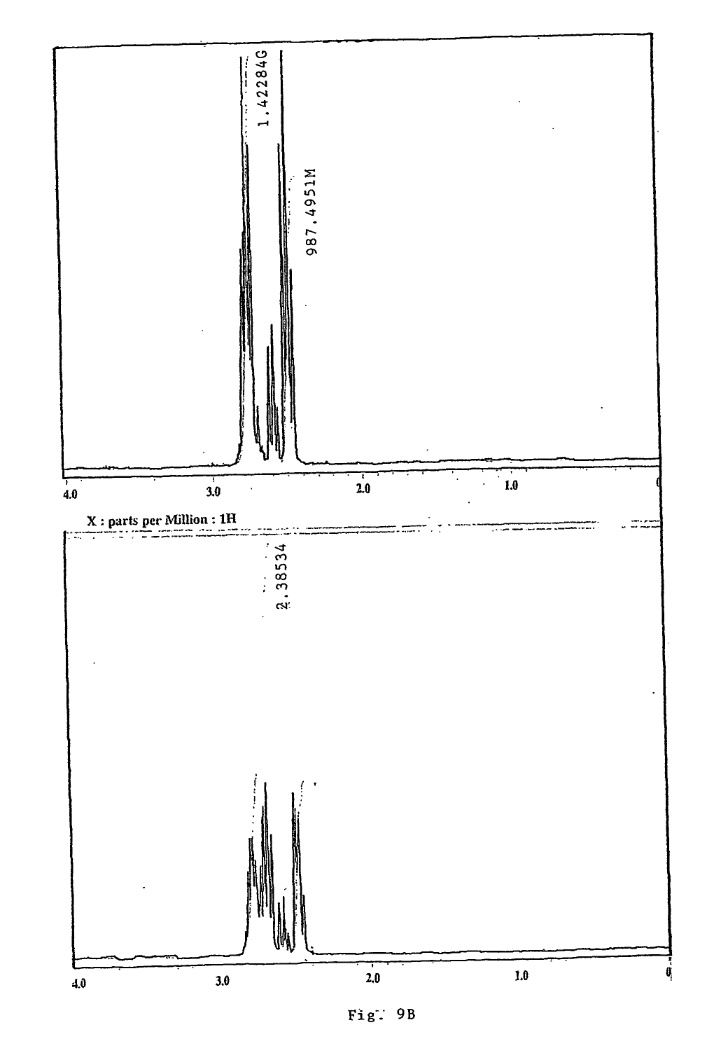

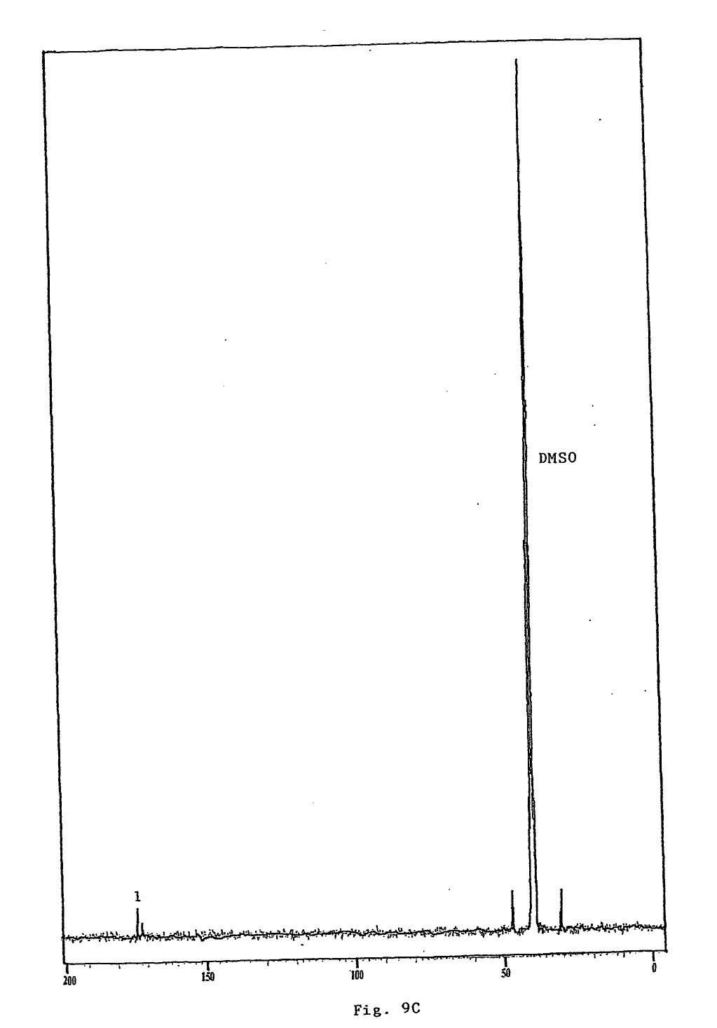

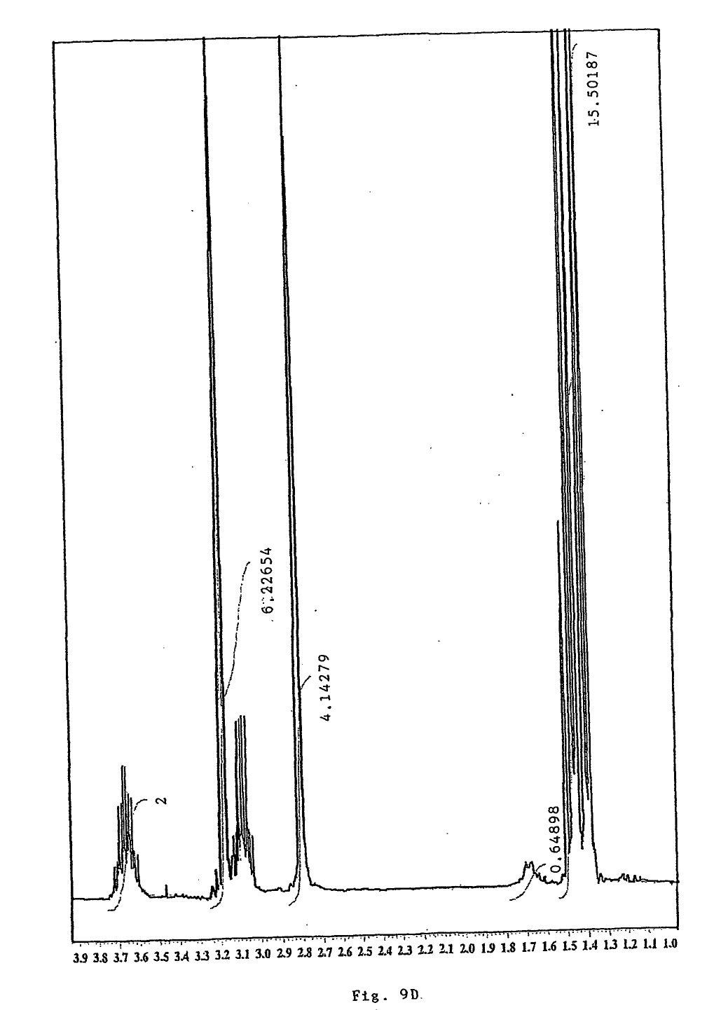

FIG. 9A-D show NMR-spectra as discussed in Example 4 below. More specifically, FIG. 9A shows the spectrum of 3-sulfopropionic acid; FIG. 9B shows the ¹³C NMR spectrum of 3-sulfopropionic anhydride, FIG. 9C shows an anhydride carbon spectrum; and FIG. 9D shows the spectrum of the NHS-ester from 3-sulfopropionic anhydride.

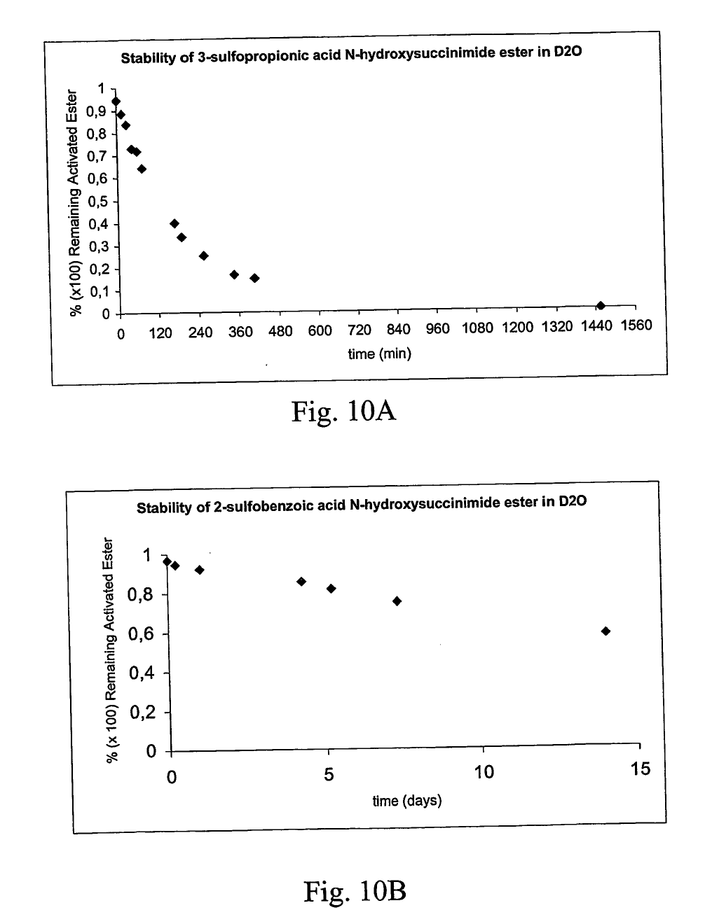

FIGS. 10A-B illustrate the stability of NHS-esters according to the invention. More specifically, FIG. 10A shows the stability of 3-sulfopropionic acid NHS-ester in D₂O while FIG. 10B shows the stability of 2-sulfobenzoic acid NHS-ester in D₂O. The analysis was conducted on a 270 MHz NMR-instrument from JEOL. NHS-ester were put in a NMR-tube and diluted with D₂O to 700 μl. A single-pulse ¹H-NMR was conducted and the spectra analysed. The hydrolysis being measured by the ratio of the integration of the signal at 2.92 ppm for 3-sulfopropionic acid N-hyrdoxysuccinimide, 3.01 ppm 2-sulfobenzoic acid N-hydroxysuccinimide and the signals of the protons of N-hydroxysucccinimide 2.76 ppm.

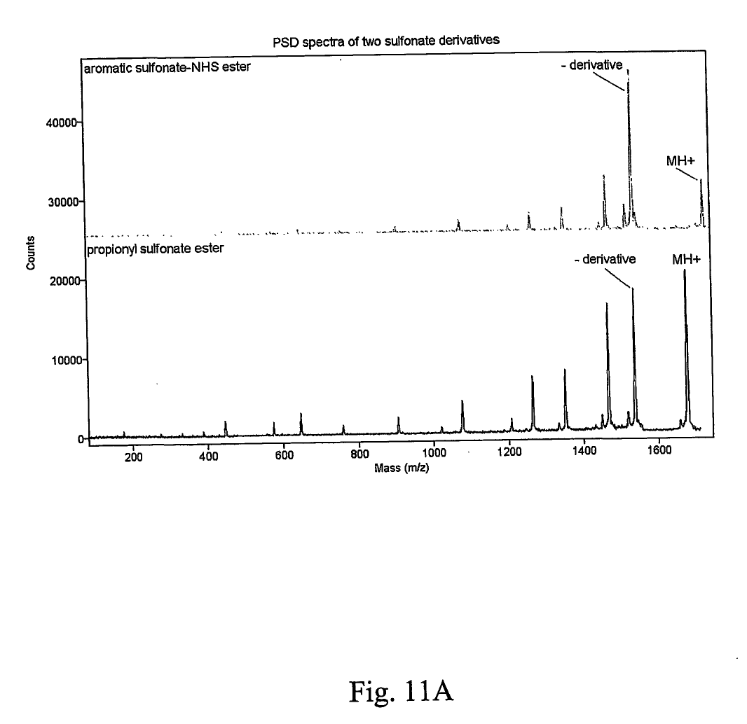

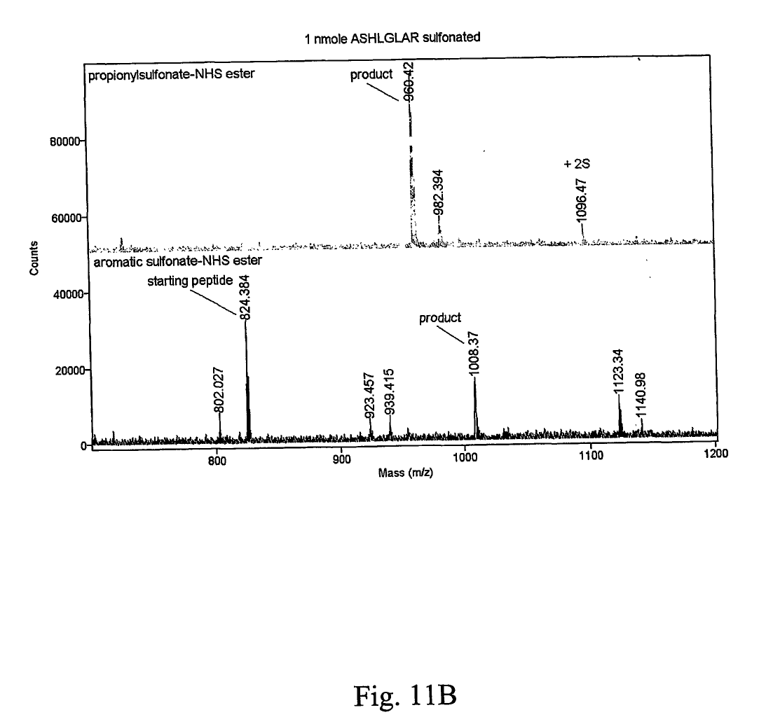

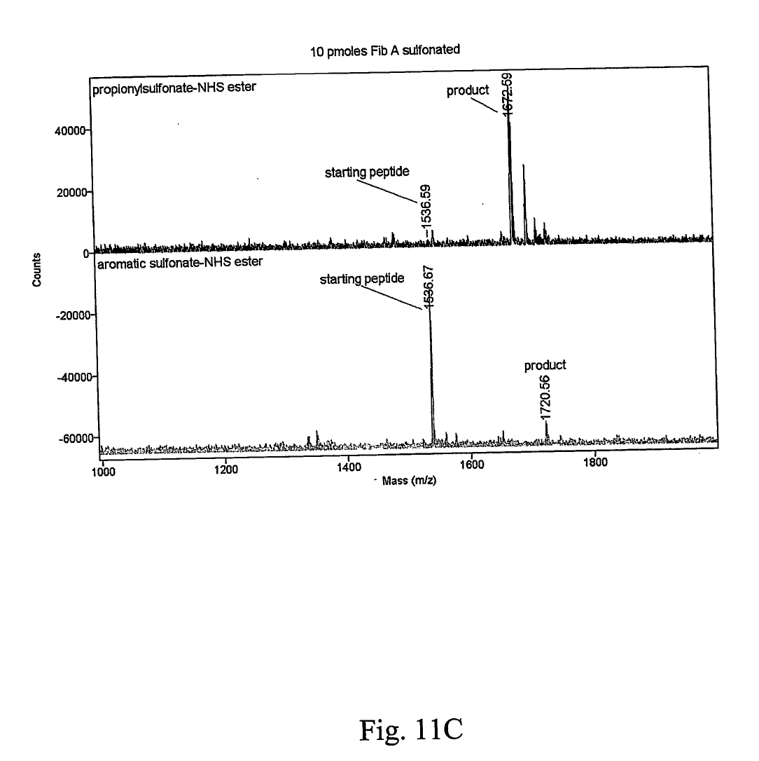

FIG. 11A-C show the MALDI PSD mass spectra produced from these derivatives and the comparative reactivities of peptides sulfonated as described in Example 7. More specifically, FIG. 11A shows a comparison of the fragmentation patterns produced from peptides containing 2-sulfobenzoic acetamides (upper) and 3-sulfopropionamides (lower). 3-Sulfopropionamides are preferred because of less loss of the derivative (which regenerates the starting peptide and is uninformative) and better yields of lower mass fragments, FIG. 11B shows a comparison of the reactivities of propionyl sulfonate NHS ester (upper) and the 2-sulfobenoic acid NHS ester (lower) with 1 nMole of a model peptide. The 3-sulfopropionic acid NHS ester shows better conversion of starting peptide to final product, and FIG. 11C is as in FIG. 11B but the reaction used 10 pmoles of FibA as the model peptide.

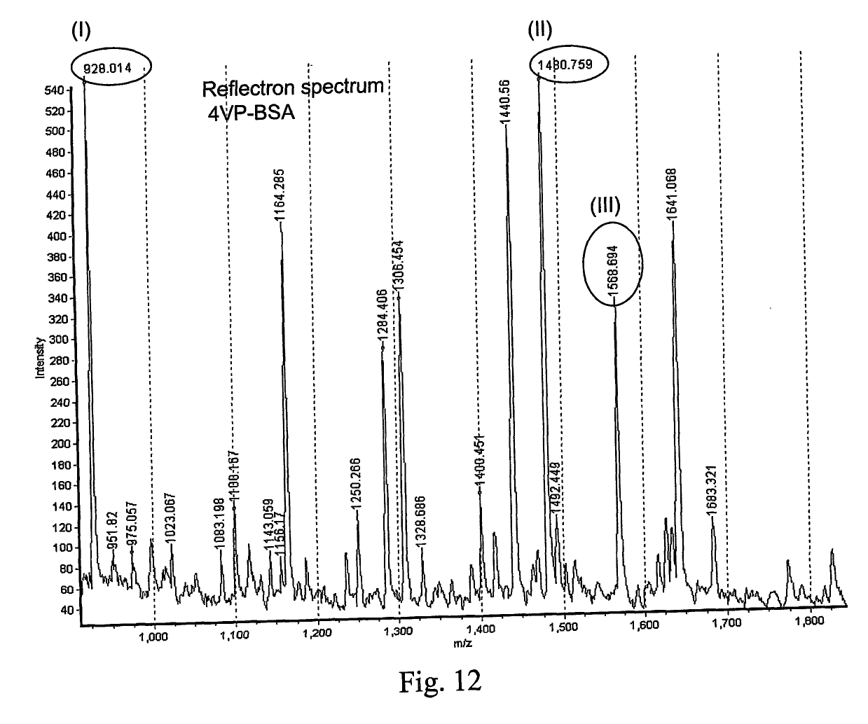

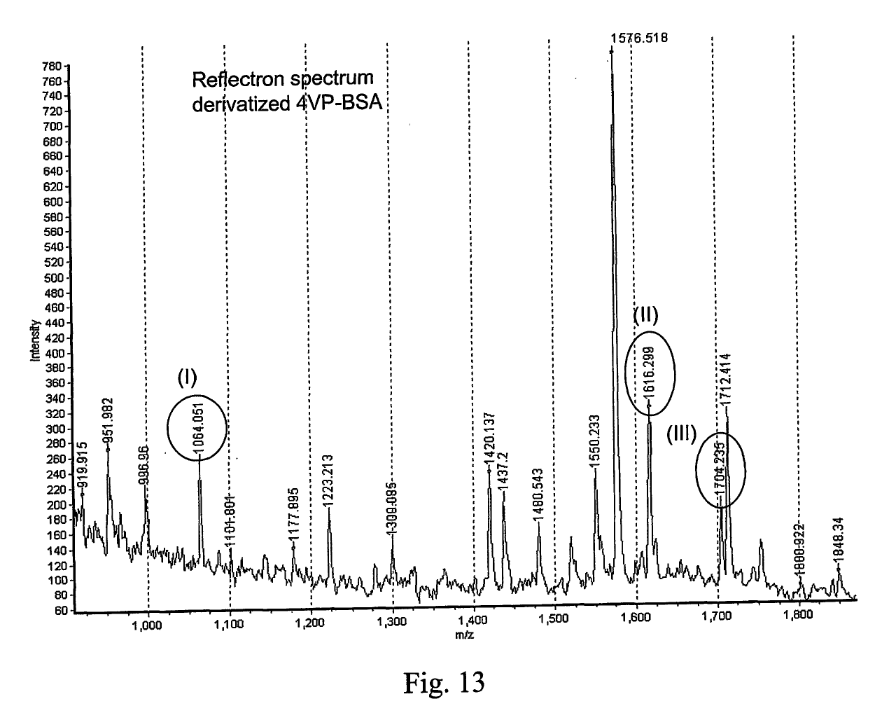

FIG. 12 shows a reflectron spectrum, positive mode (showing average masses, after filtration, smoothing 5) of 250 fmols of a non-derivatized tryptic digest of 4VP-BSA obtained with the Ettan™MALDI-TOF. (Peptides I-III were quantitatively derivatized after reaction with 3-sulfopropionic acid anhydride NHS-ester, see FIG. 13).

FIG. 13 shows a reflectron spectrum (showing average masses, after filtration, smoothing 5) of a derivatized tryptic digest of 4VP-BSA (Ettan MALDI-ToF™). The peptides were derivatized with 3-sulfopropionic acid NHS ester using aqueous conditions as described. The peptides marked I-III were quantitatively derivatized and used for PSD analyses.

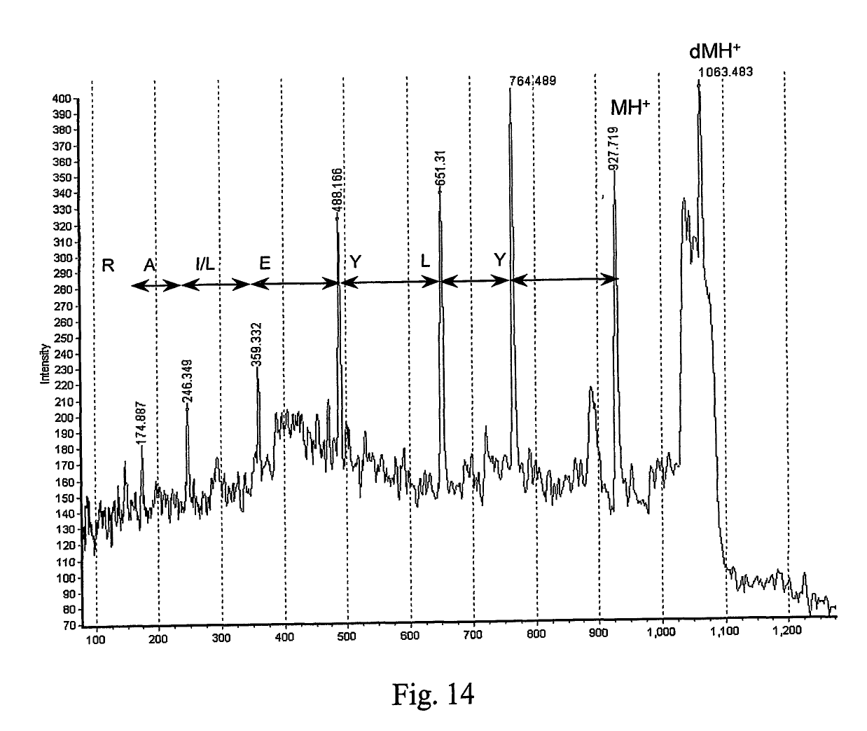

FIG. 14 shows a PSD spectrum (positive mode) showing a complete y-ion series of peptide (I) from the derivatized tryptic digest of 4VP-BSA (FIG. 13) obtained with the Ettan™MALDI-TOF. The ion gate was set on the mass of the derivatized parent ion, m/z064, and the signals from 300 shots were accumulated.

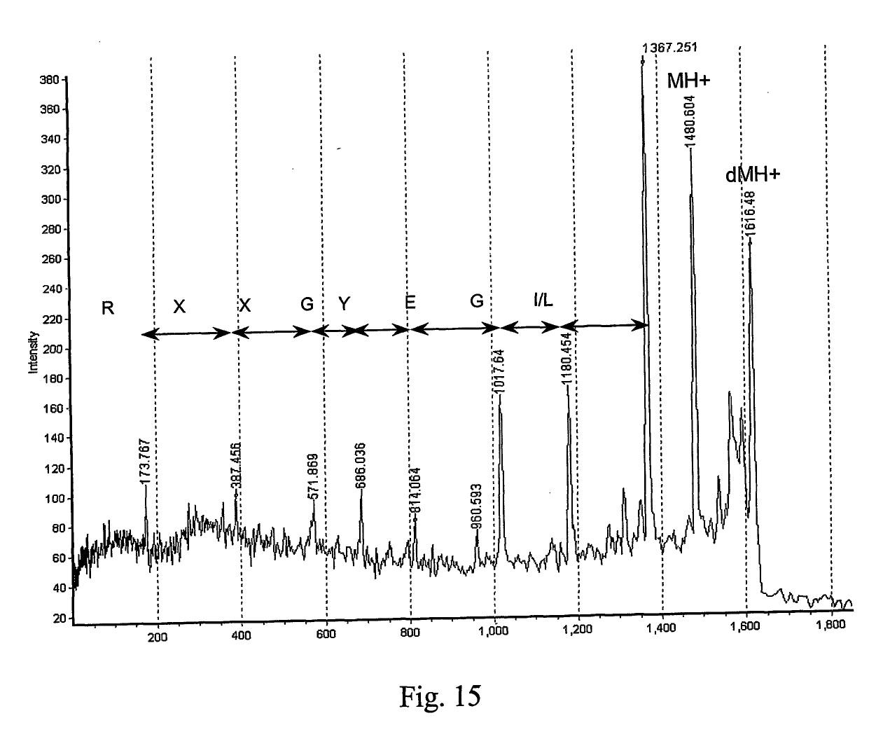

FIG. 15 shows a fragmentation spectrum (PSD, positive mode) of peptide (II) from the derivatized tryptic digest of 4VP-BSA (FIG. 13). The ion gate was here set on m/z 1616. Signals from 300 shots were accumulated. Gaps are marked with an X.

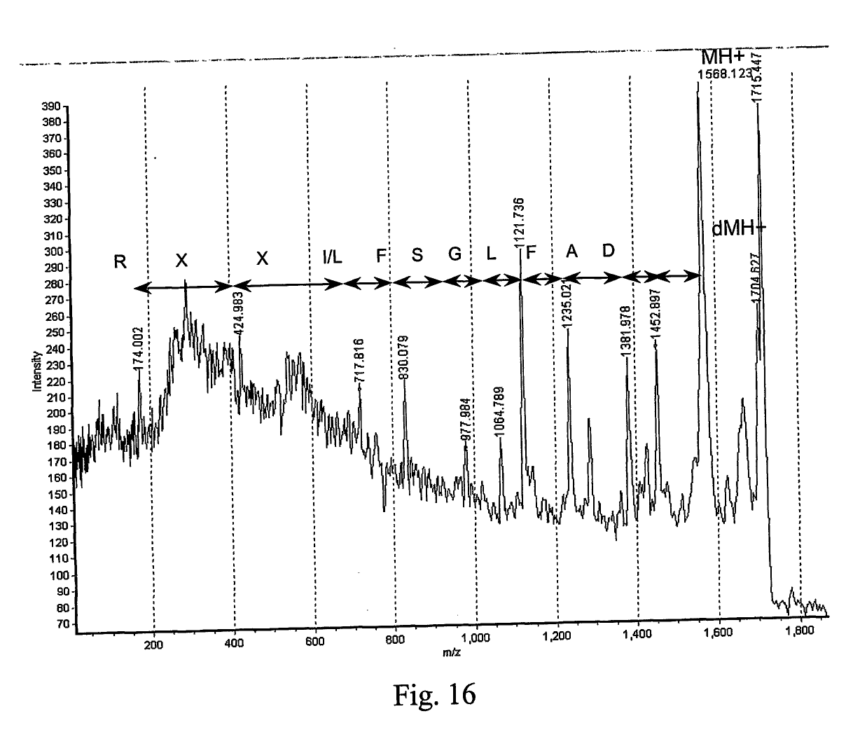

FIG. 16 shows a PSD spectrum (signals from 300 shots accumulated) of peptide (III) (FIG. 13), m/z 1704, from the derivatized tryptic digest of 4VP-BSA. Gaps are marked with an X. The peptide, MH⁺ m/z 1715, passed the ion gate together with derivatized peptide.

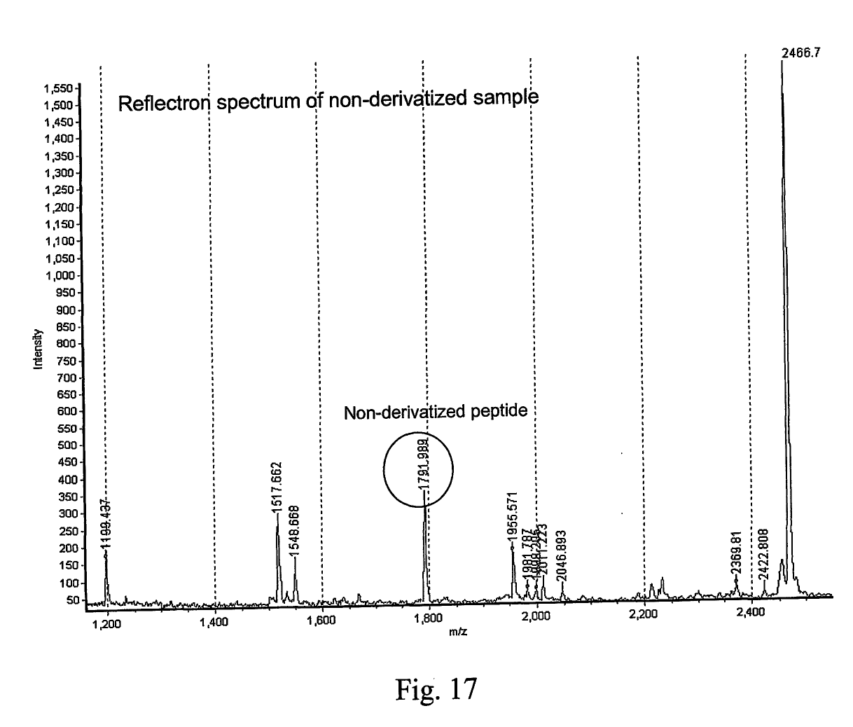

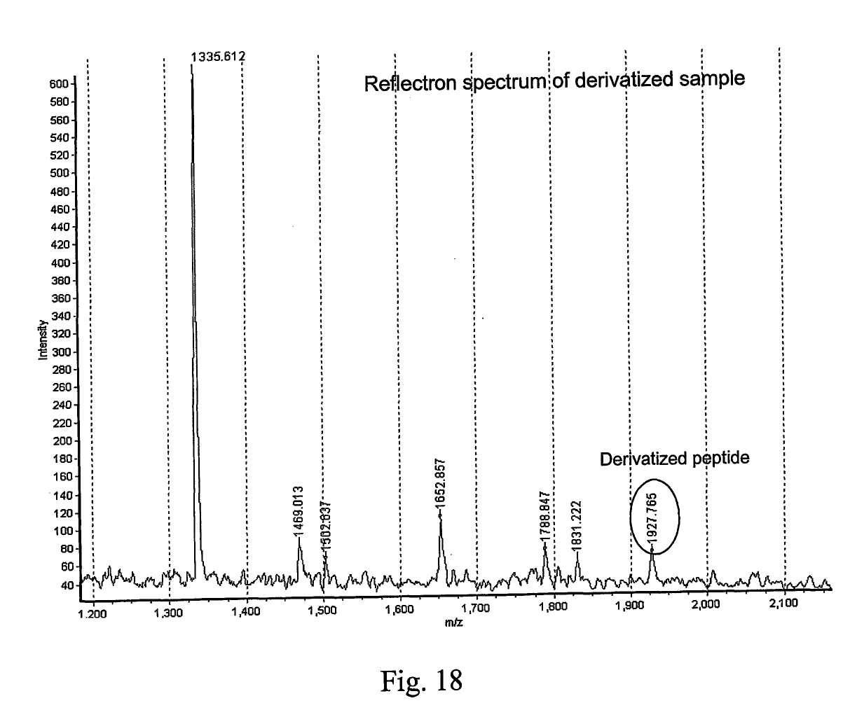

FIG. 17 shows a first example of a reflectron spectrum (positive mode, 100 shots accumulated, showing average masses, after filtration, smoothing 5) of a non-derivatized protein digest from a Coomassie-stained 2-D gel obtained with the Ettan MALDI-TOF. Five percent of the total eluted tryptic digest was used to obtain this spectrum. (The peak marked with a circle can be seen fully derivatized in FIG. 18.)

FIG. 18 shows a reflectron spectrum (positive mode, showing average masses, after filtration, smoothing 5) of the same 2-D sample as in FIG. 17 (remaining 95%), but after N-terminal derivatization with NHS-ester. The sample was cleaned up on a μC₁₈ ZipTip™, and derivatized according the protocol. The peptide m/z 1791 (previous figure) was quantitatively derivatized and is here observed with the extra mass of the label, m/z 1927.

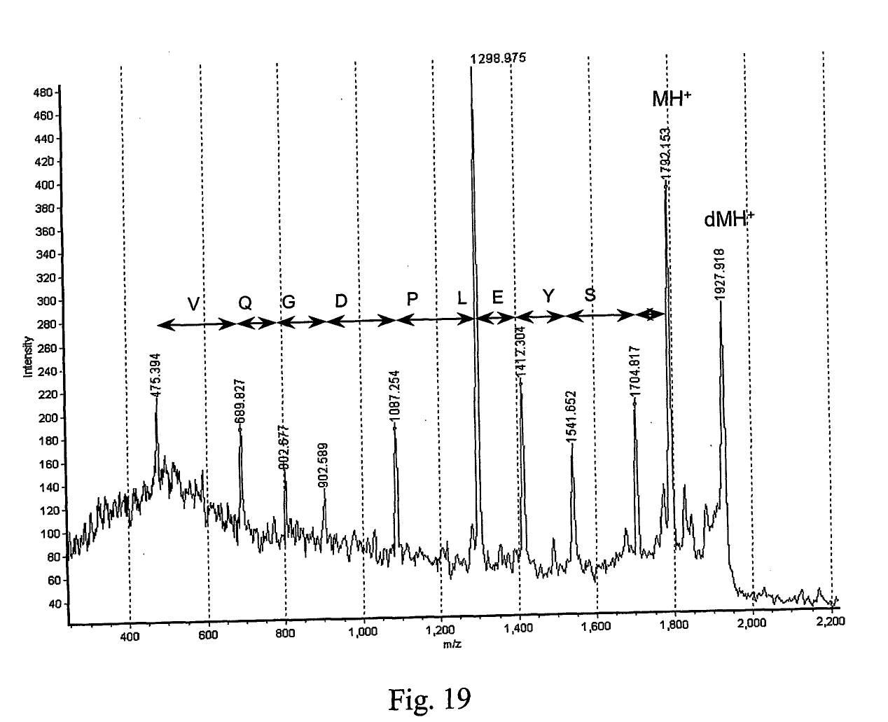

FIG. 19 shows a PSD spectrum (accumulated from 300 shots), of the derivatized peptide, m/z 1927. The masses of the fragments (y-ions) were used for identification in PepFrag. The protein was identified as actin.

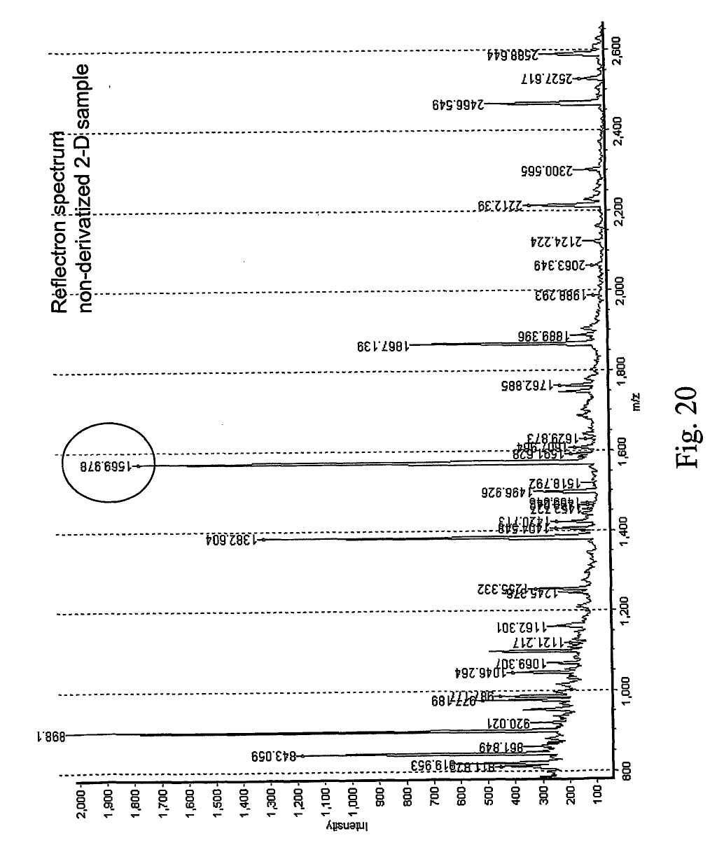

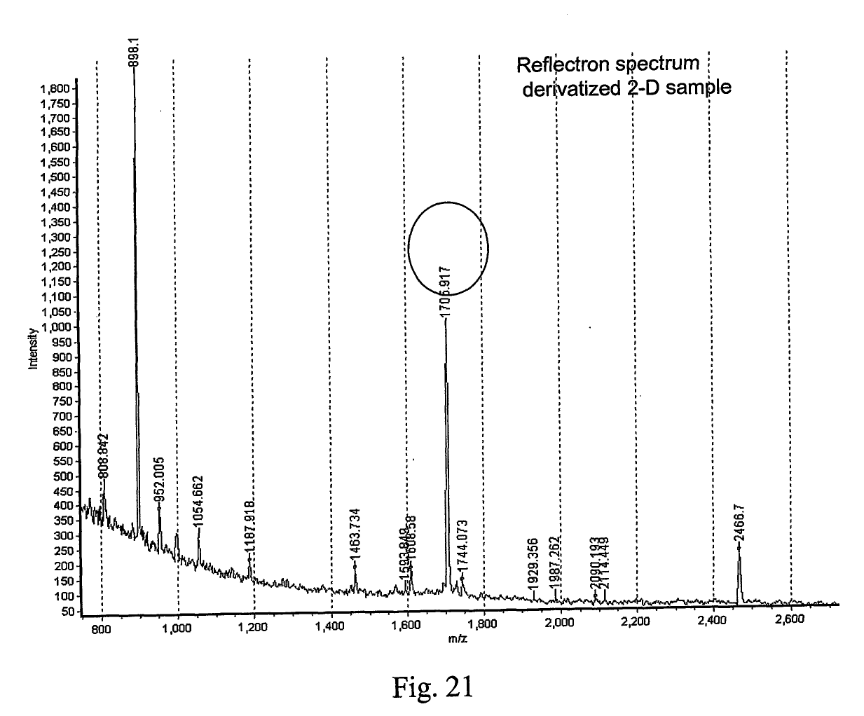

FIG. 20 shows a second example of a reflectron spectrum (accumulated from 100 shots, showing average masses, after filtration, smoothing 5) of a non-derivatized tryptic digest of a protein spot from a Coomassie-stained 2-D gel, obtained with Ettan™MALDI -TOF. Five percent of the sample was used in this analysis. The marked peptide was used for PSD analyses after derivatization (see FIG. 21).

FIG. 21 shows a reflectron spectrum (positive mode showing average masses, after filtration, smoothing 5) of the same 2-D sample as in FIG. 19, but after ZipTip™ clean up and derivatization with NHS-ester in aqueous solution as described. The peptide m/z 1569.9 (FIG. 20) was quantitatively derivatized and is here observed with the extra mass of the label (+136) as m/z 1705.9.

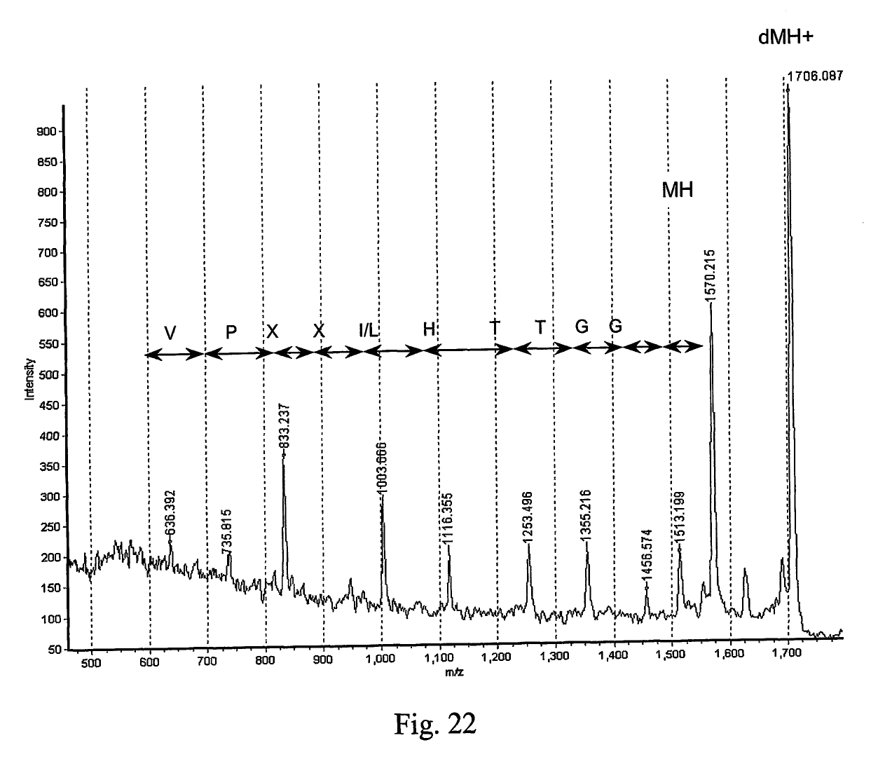

FIG. 22 shows a PSD spectrum (signal from 300 shots accumulated) of the derivatized peptide, m/z 1705 (see FIG. 20). The fragment masses (y-ions) were used for protein identification in PepFrag. The protein was identified as E-coli succinyl-CoA synthetase.

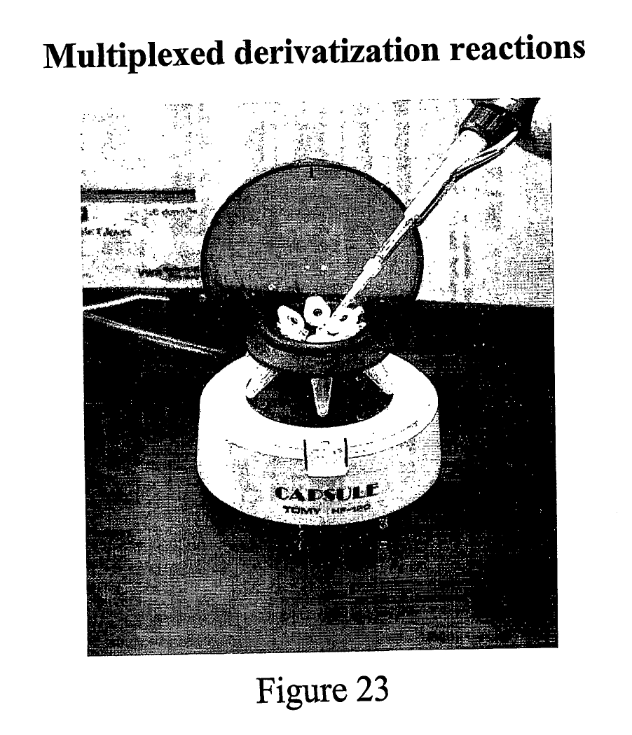

FIG. 23 shows sample-loaded ZipTips™ placed into a laboratory centrifuge for subsequent sulfonation in a multiplexed fashion.

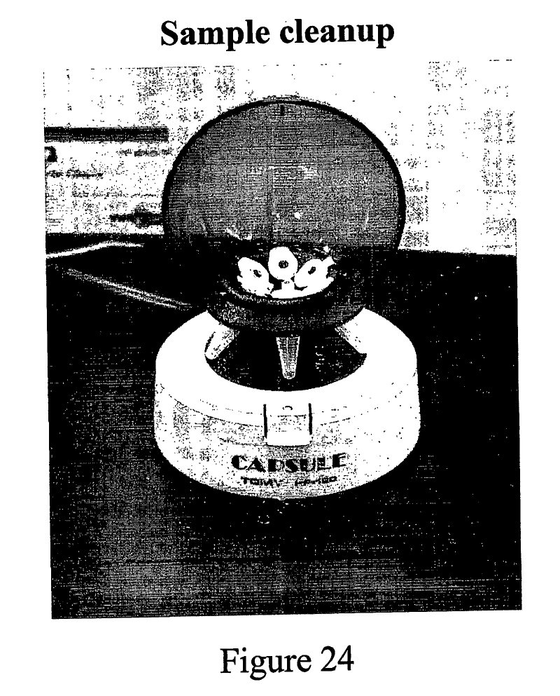

FIG. 24 illustrates sample washing in the centrifuge following the sulfonation reaction.

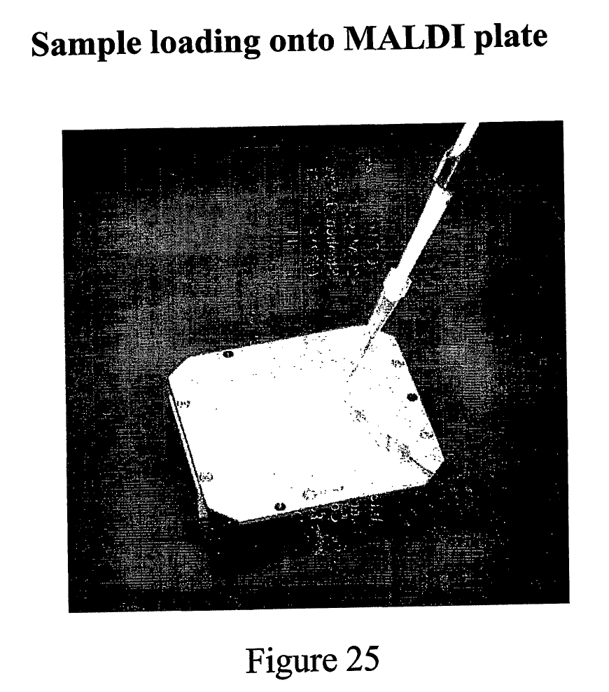

FIG. 25 illustrates direct loading of the derivatized samples from the solid supports onto the MALDI sample stage.

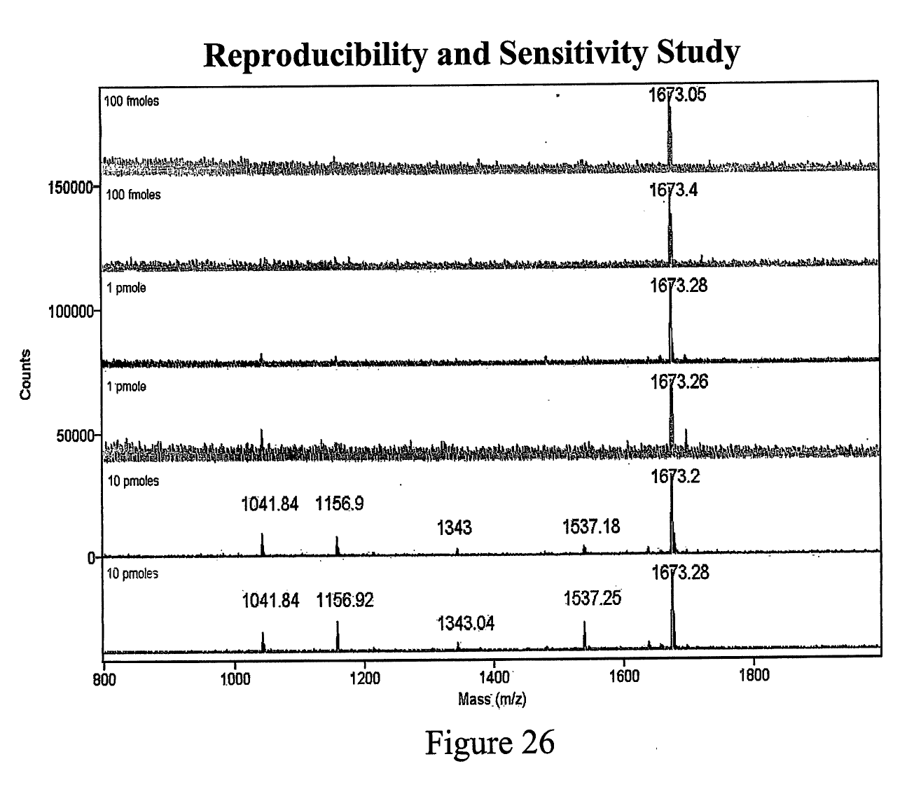

FIG. 26 shows the MALDI mass spectra obtained following sulfonation of Fibrino-peptide A on solid support. Duplicate samples were sulfonated at three different peptide levels (10, 1 and 0.1 pmoles).

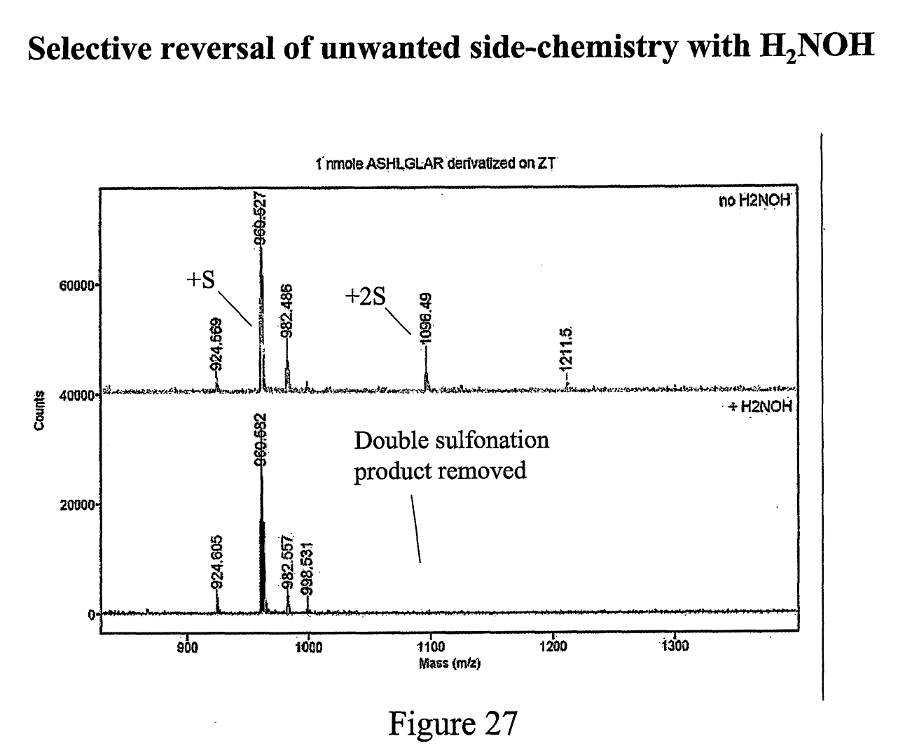

FIG. 27 the use of hydroxylamine hydrochloride for reversing unwanted ester side-products formed in the sulfonation reaction. The upper spectrum was obtained from ASHLGLAR sulfonated on solid support in the centrifuge. The lower spectrum was obtained from the same sulfonated peptide following treatment with hydroxylamine hydrochloride.

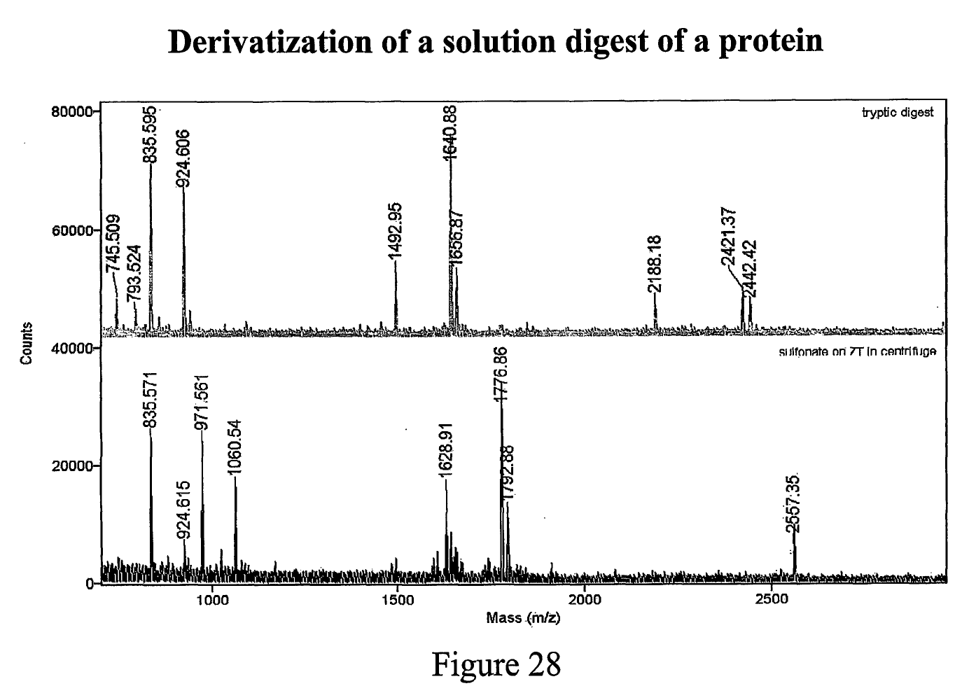

FIG. 28 demonstrates the sulfonation of a protein digest. The upper spectrum was obtained from the native protein digest. The lower spectrum was obtained following sulfonation of the digest.

Experimental Part

The present examples are intended for illustrative purposes only and should not be construed as limiting the invention as defined by the appended claims. All references given below and elsewhere in the present application are hereby included herein by reference.

### EXAMPLE 1

### Sulfonation on Solid Support, General Scheme

Reagent: 3-sulfopropionic acid N-hydroxysuccinimide ester

Buffers and Chemicals:

O-methylisourea hydrogen sulfate

0.25M NaHCO₃, pH 11.9

0.25M NaHCO₃, pH 9.4

50% hydroxylamine solution/1 μl of a 15M solution

Acetonitrile (ACN)

Trifluoracetic acid (TFA)

matrix for MALDI-TOF analyses of α-cyano-4-hydroxycinnamic acid

Buffers and solutions prepared from deionized 18.2 MΩ (DI) water

C₁₈ ZipTip™ (ZT) from Millipore (μC₁₈ ZipTips can alternatively be used)

General Procedure:

The sample can be dried down and reconstituted in 10 μl 0.1% TFA. Alternatively, the sample is dried down to about 20 uL, in which case the samples are made acidic before loading onto ZipTips.

Solid support in the form of C₁₈ ZipTip™ (ZT) is activated with 50% ACN;0.5% TFA and the ZipTip™ is then equilibrated with 0.1% TFA. A sample comprising tryptic peptides is loaded the sample on the ZipTip™ (pipett 10 times slowly up and down).

In a separate vessel, 2 μl O-methylisourea hydrogensulfate solution (86 mg/ml MQ H₂O) is mixed with 8 μl 0.25M NaHCO₃, pH 11.9. The resulting mixture is loaded on the ZipTip™ (pipett ˜5 times up and down). The tip is removed with solution on the top and put in an eppendorf tube, the lid is closed and it is placed in a heating block at 37° C. for 2 h.

The tip is then washed with 0.1% TFA (pipett ˜5 times up and down).

The sulfonation reagent solution is made fresh just prior to incubation and dissolved in 0.25 M NaHCO₃, pH 9.4 (10 mg/100 μl).

Then, step (a) of the present method is performed by passing the sulfonation reagent solution through the ZipTip™ by pipetting up and down 10 times. The solution is left on the tip for at least 3 minutes. If the reactions are being performed manually using a single-position micropipetter it may be convenient to take the tip, with solution on the top of the C₁₈ column, off of the micropipetter and set it aside. It is then possible to continue with the next sample, while waiting for completion of step (a).

In order to reduce the amount of unwanted sulfonation of internal amino acids, 1 μl 15M hydroxylamine solution is added to the reagent solution. Mix and load to the ZT and pipett up and down 10 times. In the alternative embodiment, a small volume of the hydroxylamine solution is passed over the ZTs containing the sulfonated peptides. Thus, in this last mentioned embodiment, the hydroxylamine is never with the original reagent solution.

The ZT is the preferably washed with 0.1% TFA and the sample is eluted in 10 μl 80% acetonitrile:0.5% TFA.

To analyze the derivative(s) obtained, the sample is dried down and reconstituted in 3 μl, 0.1% TFA. A total drying in this step will allow a more exact analysis, since it compensates for differences in sample volumes by standardising the procedure, which is especially desired in automated procedures. The sample is mixed 1:1 with saturated alpha-cyano-matrix solution in 50% ACN:0.5%. The sample is then loaded on the MALDI target and analyzed.

As mentioned above, in one embodiment, which is especially suited for low-level analytes, the samples are not dried down. The cleaned up products are then eluted off of the ZT directly onto the MALDI sample plate, for example using 2.5 uL of 50% ACN:0.5% TFA containing the MALDI matrix. This way, sample handling losses are reduced and preferably avoided altogether, so that all of the products can be transferred to the MS.

### EXAMPLE 2

### Guanidination and Sulfonation of a Low Level Tryptic Digest of Horse Myoglobin Immobilized to Solid Support

Alkylation and Trypsin Digestion of the Protein:

Horse myoglobin (Sigma) was dissolved in MQ water to a concentration of 1 μg/μl and 50 μl was mixed with 450 μl denaturation buffer (8 M UREA, 50 mM TRIS-HCl pH 8.0, 50 mM DTT (all chemicals were plusone™)) and incubated for 1 hour at 37° C., in order to denature the protein and disrupt any disulfide bonds. The cysteine SH groups were then chemically blocked by 2-Iodoacetamide (MERCK), by adding 500 μl alkylation buffer (8 M UREA, 50 mM TRIS-HCl pH 8.0, 125 mM 2-Iodoacetamide). The reaction was allowed to proceed for 1 hour at 37° C., The sample was thereafter purified on a NAP-10 column, equilibrated with 15 ml 10 mM NH₄HCO₃. The sample was applied (1000 μl) and eluted in 900 μl 10 mM NH₄HCO₃. The protein was digested by adding 5 μg of trypsin (Promega, V511A) to the eluted sample. The trypsin digestion reaction was left over night (approximately 14 hours) at 37° C., and terminated by the addition of 5 μl of concentrated trifuoroacetic acid (TFA) (Pierce) to a final concentration of 0.5%. The digested sample was diluted stepwise in 0.1% TFA to a fmal concentration of 15 fmol/μl. The resulting material was stored at −20° C.

Guanidination and Sulfonation on Solid Support:

A C₁₈ ZipTip™ (Millipore) (ZT) was activated with 50% acetonitrile;0.5% TFA (by pipetting 2 times up and down). The ZT was thereafter equilibrated with 0.1% TFA (by pipetting 2 times up and down). Tryptic digest of horse myoglobin (150 fmol in 10 μl 0.1% TFA) was loaded to the ZT (pipett 10 times slowly up and down). A stock solution of O-methylisourea (84 mg/ml MQ H₂O) was prepared. Two microliters of the stock solution of O-methylisourea was mixed with 8 μl 0.25 M, NaHCO₃ buffer, pH 11.7 and the solution was loaded to the ZT. The ZT was left in a closed eppendorf tube in 37° C. for 2 h, for the sample to react. The ZT was therefore washed with 10 μl 0.1% TFA (by pipetting 2 times up and down). NHS-ester of 3-sulfopropionic acid anhydride, was dissolved in 0.25 M NaHCO₃ buffer, pH 9.4, to a final concentration of 100 mg/ml. Ten μl of the NHS-ester solution was loaded to the ZT. The sample was left to react for 3 minutes in RT. One microliter of 15M hydroxylamine solution was added to the NHS-ester reagent and loaded to the ZT (by pipetting 5 times up and down).

The tip was washed with 0.1% TFA and the sample eluted in 10 μl 80% acetonitrile:0.5% TFA. The sample was dried down under nitrogen and reconstituted in 3 μl 50% acetonitrile. 0.3 μl of the sample was loaded to the MALDI target, using the Ettan MALDI spotter and mixed with 0.3 μl saturated α-cyano matrix solution. The sample was analyzed in reflectron and PSD modes using the Ettan MALDI ToF.

One-tenth of the 150 fmole tryptic digest of horse myoglobin, which was guanidinated and sulfonated following immobilization onto a ZipTip™, was analyzed using the Ettan MALDI ToF. For comparison, FIG. 1 shows the reflectron spectrum of a non-derivatized sample of horse myoglobin (15 fmol on MALDI target) and FIG. 2 the reflectron spectrum of derivatized sample (<15 fmol on the MALDI target). Due to the efficient guanidination of the lysines on solid support the signals for the lysine-terminated peptides were dramatically increased in the reflectron spectrum of derivatized sample compared to the analysis of the non-derivatized sample. Two derivatized peptides were used for PSD analysis (one lysine terminated peptide m/z, 1449.5 and one arginine terminated peptide m/z, 1742.8). FIG. 3 shows the PSD spectrum of m/z 1449.5. The protein was identified in PepFrag, by submitting the observed y-ion masses (−42 Da mass increment from the guanidination reaction) FIG. 4. FIG. 5 shows the fragmentation spectrum of an arginine-terminated peptide (m/z 1742.8). The eight y-ions obtained were used for protein identification in PepFrag (FIG. 6).

The guanidination and sulfonation reaction times are reduced when the reactions are carried out with peptides or polypeptides immobilized to a solid support. The overall efficiency of the derivatization procedures is improved, and better sensitivity results because dilute analyte solutions can be concentrated prior to reaction and because reduced sample losses occur as a result of reduced sample manipulation prior to analysis. The example shows protein identification by derivatization PSD analysis, starting with as little as 15 fmol of the protein.

### EXAMPLE 3

### Alternative Method to Sulfonate Peptides and Polypeptides Immobilized to a Solid Support

Peptides and polypeptide mixtures in solution are concentrated to a final volume between 10 to 50 μl. The pH of each solution is made acidic, and the peptide/polypeptide solutions are loaded onto C₁₈ ZipTips. The sample-loaded Zip-Tips™ are placed into the tops of drilled-out, closed microcentrifuge tubes, which are loaded into a laboratory centrifuge as shown in FIG. 23. The sample-loaded tips are washed with 0.1% TFA. This. is accomplished by adding 25 μl of 0.1% TFA to the tops of each tip and spinning. The centrifugal force is sufficient to move the solution over the tip. The solution is collected into the bottom of the microcentrifuge tube. This wash step is repeated two more times. Samples are then sulfonated using e.g. propionylsulfonate-NHS ester. The sulfonation reagent is prepared at a concentration of 10 mg/100 μl base (H₂O:DIEA 19:1 v:v) just prior to use. The pH of the reagent solution is checked, and adjusted if necessary, to be sure that it is basic prior to use. The samples are sulfonated by loading 5 μl of the sulfonation solution to the top of each sample-loaded tip. The samples are spun again to transport the sulfonation reagent over the tips. All samples in the centrifuge are sulfonated in parallel using this procedure. Optionally, the sample-loaded tips can be further treated with hydroxylamine hydrochloride to reverse any unwanted ester side-products that may have been formed during the sulfonation step. That reaction is carried out by loading 5 μl of fresh hydroxylamine hydrochloride solution (2M in H₂O:DIEA 19:1 v:v, pH adjusted to basic prior to use) to the top of each sample-loaded tip. The samples are again spun to transport that solution over the tips. The samples are then washed three times with 25 μl of 0.1% TFA, as shown in FIG. 24. The derivatized samples are loaded directly from the ZipTips™ onto a MALDI sample stage for analysis. The samples are eluted onto the sample stage with a small volume (2.5 μl of ACN:0.1% TFA (1:1 v:v) containing 10 mg/ml of a suitable MALDI matrix like α-cyano-4-hydroxycinnamic acid or 2,5-dihydroxybenzoic acid, as shown in FIG. 25.

The utility of this approach is illustrated with data presented in the next few figures. For example, FIG. 26 shows the MALDI mass spectra obtained from varying quantities of Fibrinopeptide A (ADSGEGDFLAEGGGVR) sulfonated according to the method just discussed. The starting MH⁺ mass of Fib A is 1536.7 and the desired monosulfonate product weighs 1672.7 Da. The measured molecular masses are in error about 0.5 Da because the mass scale was not accurately calibrated in these experiments. The spectra indicate near quantitative sulfonation even at the 100-fmole level. Note that the lower mass ions in the 10-pmole samples (lower two traces) result because too much sample was presented to the mass spectrometer in those two analyses. The ions having masses less than that of the sulfonation product mainly result from fragmentation processes that occurred within the ion source during analysis. FIG. 27 compares MALDI mass spectra of a small Arg-terminated peptide (ASHLGLAR), which was sulfonated as just described. The top spectrum in the figure was obtained following sulfonation. It shows signals for the desired product at about m/z 960, and a signal for an unwanted double sulfonation product at about m/z 1096. The lower spectrum was obtained from the same sulfonated peptide after treatment with hydroxylamine hydrochloride as described above. Note that the unwanted sulfonation product at about m/z 1096 has been greatly reduced in relative abundance. The spectra in FIG. 28 demonstrate that protein digests can also be efficiently sulfonated using this method. The upper spectrum in the figure was obtained from the native tryptic digest, which was not sulfonated. The lower spectrum was obtained from the protein digest that was sulfonated according to the present method. The peptide masses observed in the top spectrum shift upwards by 136 Da following sulfonation according to the present method. Near quantitative sulfonation of the protein digest was observed in this experiment.

### EXAMPLE 4

### Comparative: Sulfonation in Solution vs on Solid Support

Sulfonation in Solution

General Method

The sample (BSA tryptic peptides) was dissolved in 5 μl of water. 10 μl of 20% DIEA solution was added followed by 5 μl of NHS ester solution. After 15 minutes, hydroxylamine was added to hydrolyse unwanted ester groups, which may have been formed during the sulfonation step. The pH of the resulting solution was made acidic (<4) by addition of 50% TFA. The reacted peptides were bound to reverse phase chromatography (RPC) solid support (ZipTip™, Millipore) and eluted using 80% Acetonitrile and 0.5% TFA. The eluted sample was dried and reconstituted in 3 μl of 50% ACN, 0.5% TFA for further analysis on MALDI.

Sample: BSA tryptic peptides

Reaction vessel: 500 μl Eppendorff tube

Total volume: 20 μl

Water: 5 μl

Volume of base: 10 μl of 20% DIEA (shake thoroughly before pipetting as it is immiscible) or 2 μl neat DIEA

Volume of NHS ester: 5 μl (10 mg/100 μl)

Reaction time: 15 minutes or more

Addition of hydroxylamine: 2 μl

Neutralization: Add 3 μl of 50% TFA to neutralize before cleaning up with ZipTip™.

Preparing ZipTip™ for binding peptides: Wet the C₁₈ matrix with 50% acetonitrile and then equilibrate with 0.1% TFA.

Elution: 80% Acetonitrile and 0.5% TFA in another tube

For making matrix: 50% Acetonitrile, 0.5% TFA

Sulfonation on Solid Support

General Method

Bind the sample (peptides having arginine or homoarginine as C-terminal) to solid support, preferably C₁₈ on chemically resistant matrix. Here we have used ZipTip™ C₁₈ 0.6 μl supplied by Millipore). Leave in contact with reaction mixture (NHS ester+base) for a minimum of 3 minutes. Add hydroxylamine to the reaction mixture, to hydrolyze any unwanted ester side-products that may have been formed during sulfonation, and aspirated it up and down five times. Wash the solid support with 0.1% TFA and elute it for further analysis.

Preparing ZipTip for Binding peptides: Wet the C₁₈ matrix with 50% acetonitrile and then equilibrate with 0.1% TFA

Sample: BSA tryptic peptides

Reaction vessel: 500 μl eppendorf tubes

Volume of NHS ester of propionic acid: 10 μl (10 mg/100 μl) dissolved in 0.25 M Sodium bicarbonate.

Reaction time: minimum of 3 minutes

Addition of hydroxyl amine: 1 μl

Elution: 80% Acetonitrile and 0.5% TFA in another tube

For making matrix: 50% Acetonitrile, 0.5% TFA

MALDI Analysis

For this sulfonation reaction, the intensities of five arginine peptides (see table below and FIGS. 7 and 8) were studied and compared.

TABLE 1
Peptides studied

| | | | | Tyrosine |
|---|---|---|---|---|
| Sequence | Peptide | Native | Sulfonated | labeled |
| 347-359 | DAFLGSFLYEYSR | 1567.8 | 1703.8 | 1839.8 |
| 421-433 | LGEYGFQNALIVR | 1479.9 | 1615.9 | 1751.9 |
| 360-371 | RHPEYAVSVLLR | 1439.9 | 1575.9 | 1711.9 |
| 361-371 | HPEYAVSVLLR | 1283.7 | 1419.7 | 1555.7 |
| 161-167 | YLYEIAR | 927.49 | 1063.49 | 1199.49 |

Results

See discussion in relation to FIGS. 7 and 8 above.

Comparison of Results from Reactions Performed in Solution and on Solid Phase

1. The reaction time in solid phase was about 3 minutes where as it was 15 minutes for the solution.

2. When sodium bicarbonate solution was used in solution phase, very high signal to noise ratio was observed on the spectra, whereas in solid phase there was no effect on the baseline.

3. A thorough mixing of solution is required when DIEA is used as base in liquid phase.

4. As seen from FIG. 7 and 8 that spectra of 500 fmole on solid phase and 4.5 picomole of BSA peptides in solution had comparable sensitivity on MALDI.

### EXAMPLE 5

### Preparation of 3-sulfopropionic acid N-hydroxysuccinimide ester

Materials

Chemicals for Synthesis:

N-Hydroxysuccinimide (NHS), internal supply, Art-Nr 30070800

3-Mercaptopropionic acid from ALDRICH 99+%, CAS-107-96-0

Hydrogen peroxide (30%, aqueous solution)

Acetic acid (glacial) 100% from KEBO CAS-64-19-7

Potassium hydroxide from Merck, pellets

n-Heptane from Merck 99%

Thionyl chloride from ALDRICH 99+%, CAS-7719-09-7

n-Hexane from Merck 99%

Diisopropyl amine from ALDRICH 99%, CAS-7087-68-5

Dichloromethane from ALDRICH 99.8% anhydrous, CAS-75-09-2

Argon gas-tube from Air Liquide

Ethyl acetate from KEBO, CAS-141-78-6

Methanol from KEBO, CAS-67-56-1

TLC Silica gel 60 F₂₅₄ on plastic sheets from Merck

Chemicals for Analysis:

Chloroform-d from Cambridge Isotope Laboratories 99.8%, CAS-865-49-6

Deuteriumoxide (D₂O) from Larodan Fine Chemicals CAS-7789-20-0

Methods

NMR-analysis:

The analysis was conducted on a 270 MHz NMR-instrument from JEOL.

10 mg of NHS-ester were put in a NMR-tube and diluted with CDCl₃ to 700 μl . A single-pulse ¹H-NMR was conducted and the spectra analysed. The analysis was conducted in the same way for 3-sulfopropionic anhydride. For the 3-sulfopropionic acid, D₂O was used as a solvent instead of CDCl₃.

For the 3-sulfopropionic anhydride a decoupled ¹³C-NMR was carried out in the same way as with the ¹H-NMR (see above).

Melting Point Determination:

The melting point for the NHS-ester crystals was obtained on a BÛCHI Melting Point B-540 apparatus. A few crystals were put in a vial and heated until they melted. The temperature interval was from 160° C. to 185° C. and the temperature gradient 1° C./min.

Stability Test in Water:

10 mg of NHS-ester were put in a NMR-tube and 700 μl of D₂O was added. A single-pulse ¹H-NMR was conducted and the spectrum analysed. The same sample was stored at RT (20-25° C.) and after 5 and 24 hours another ¹H-NMR spectrum was collected.

Stability Test in Air:

10 mg of NHS-ester were put in a NMR Tube and analysed as above with Chloroform-D as solvent. About 100 mg of the NHS-ester were then put in a flask and kept without lid in air and RT (20-25° C.) for some days. The hydrolysis of the ester was followed with NMR.

Synthesis:

Synthesis of 3-Sulfopropionic acid

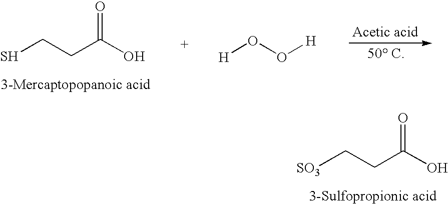

A 3-necked roundbottomed flask (500 ml) was equipped with a thermometer, dropping is funnel and a degassing pipe. A gas-trap with two security-flasks (coupled in series after each other), the last containing 25% KOH-solution was fitted to the pipe. During the reaction a nitrogen-balloon kept an inert atmosphere through the system. Acetic acid (70 ml) and hydrogen peroxide (70 g, 30% aqueous solution, 620 mmol) were put in the flask and the solution was heated under stirring to 50° C. on a waterbath 3-Mercaptopropanoic acid (8.20 ml, 94 mmol) was added very carefully through the dropping funnel over a period of about 1 hour. An exothermic reaction started at once and the temperature rose to about 80° C. The solution was then cooled on an ethanol/CO₂ bath (−72° C.) until the temperature was again 50° C., this procedure was repeated until all the 3-mercaptopropanoic acid had been added from the dropping funnel. The reaction was then left stirring at 50° C. for two hours and at RT over night.

The solvent was evaporated on a rotary evaporator (water-bath 40° C., 100 mbar) until the volume had been reduced to about 30 ml, the rest was then removed by azeotropic evaporation with 3×300 ml heptane. The resulting oil was dried in a desiccator under high vacuum over night. The crude product was a white precipitate in an oil. The yield was about 50%, estimated from the NMR-spectrum, see FIG. 1.

Synthesis of 3-sulfopropionic anhydride:

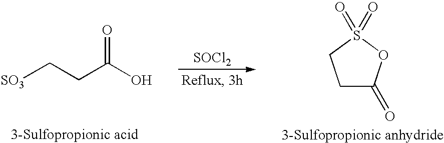

The 3-sulfopropionic acid (20 g of the crude product from the experiment above) was put in a 3-necked roundbottomed flask. A reflux-condenser and a septum were fitted to the flask. During magnetic stirring, SOCl₂ (140 ml) was carefully added through the septum over a period of 30 minutes. When all the SOCl₂ had been added the mixture was refluxed for 3 hours. Everything had dissolved during reflux into a brown-red coloured solution. After cooling for about 5 minutes, hexane (140 ml) was added. A white solid precipitated at once and a brown oil was formed at the bottom of the flask. The solution was then heated again until the white solid had dissolved and the solution was decanted into another flask to get rid of the oil. The solution was then allowed to cool in RT for an hour and then put in a refrigerator over the weekend for crystallisation.

The precipitate was filtered under nitrogen atmosphere, washed with cold n-hexane (from the refrigerator) and dried in a desiccator under high vacuum over night. All equipment that was used for the filtration had been dried in an oven beforehand and cooled in a desiccator, since the anhydride is very sensitive to water.

Synthesis of NHS-ester from 3-sulfopropionic anhydride:

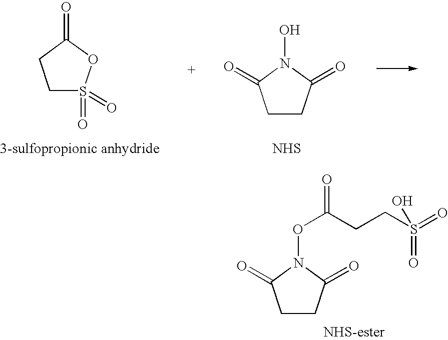

All equipment that was used was dried in an oven (100° C.) and put in a desiccator before the synthesis.

NHS (420 mg, 3.68 mmol) was weighed into a round-bottomed flask (100 ml) equipped with a septum and an argon balloon. DCM (20 ml, anhydrous 99.5%) was added and magnetic stirring began. DIEA (0.64 ml, 3.68 mmol) and 3-sulfopropionic anhydride (0.50 g, 3.68 mmol) were added carefully during stirring. The reaction was left stirring for three hours under an argon atmosphere. The solvent was evaporated (RT, 100 mbar) and the product was dried in a vacuum oven over night (RT, 1 mbar). The resulting crystals were dissolved in the minimum amount of warm EtOAc/MeOH (9:1). When everything had dissolved the solution was left to cool in RT for about three hours and then in the freezer over night. During the night white crystals had formed which were filtered on a glass filter (p3) and washed with cold ethyl acetate (5° C.). Finally the crystals were dried under high vacuum in a desiccator to get the DIEA-salt of the NHS-ester as white crystals (42% yield).

Results & Discussion

Synthesis

Synthesis of 3-Sulfopropionic acid:

The synthesis was quite simple and gave the crude 3-sulfopropionic acid as a white slurry. The tricky part was to keep the reaction at 50° C., this was done with alternating ice-bath and oil-bath which perhaps is not the most effective way. The temperature during the reaction varied from 20° C. up to 80° C. If a better temperature control could be maintained under the reaction maybe the yield would improve. No further purification was done since it was not necessary for the next step (synthesis of the anhydride) making the yield very hard to calculate. On the NMR-spectra you could see at least one bi-product and maybe some of the starting material (see NMR-analysis) an estimation of the purity would be around 50%.

Synthesis of 3-sulfopropionic anhydride:

As expected the anhydride was very sensitive to water and it was necessary to dry all equipment in an oven before use and to do the reaction and purification under an argon atmosphere. The reaction and recrystallisation was done in SOCl₂ which is a very toxic solvent. The product, 3-sulfopropionic anhydride, was collected as light-brown crystals. For a reliable calculation of the yield, it is essential that the starting material is pure.

Synthesis of NHS-ester from 3-sulfopropionic anhydride:

Once again the equipment was dried in an oven before the reaction which was done under an argon atmosphere. The reaction was quite simple and after two hours of stirring the solvent was evaporated to give the crude NHS-ester/DIEA-salt as a white/yellow solid. The yield after purification was 42%. A longer reaction time and excess NHS and/or DIEA could possibly improve the yield. The yield is also calculated on a 100% pure 3-sulfopropionic anhydride.

Purification:

The crude NHS-ester/DIEA-salt was recrystallized. This was done in EtOAc/MeOH (9:1) after first trying EtOAc/MeOH (7:3). The latter one gave no crystallisation after cooling.

In the synthesis of the anhydride (see above) a sort of recrystallisation was done in SOCl₂. This however was in reality just a re-heating of the reaction mixture and a decantation to get rid of the oil in the bottom of the flask. A better purity of the anhydride will be achieved by a proper recrystallisation.

Characterisation

Melting Point Determination:

The melting point of the crude NHS-ester/DIEA-salt was between 145-155° C. After recrystallisation however the melting point was determined to 176-178° C., This higher and much sharper melting point after purification indicates that the product has indeed become purer.

NMR-Analysis:

The spectra obtained from NMR analysis is shown in FIG. 1.

3-sulfopropionic acid:

TABLE 2
Interpretation of the ¹H-NMR-spectra of
3-sulfopropionic acid CDCl₃

| | shift (δ | | |
|---|---|---|---|
| Proton number | ppm) | Interpretation | Group |
| 1, 2 | 3.13 | t, methylene protons | O₃S—CH2—CH2— |
| | | | COOH |
| 3, 4 | 2.75 | t, methylene protons | CH₂—CH2—COOH |

The spectra also contained some by-product and some starting material giving some peaks at δ2.78, δ2.85, δ3.18 and at δ3.52. This was expected when no purification had been done.

3-Sulfopropionic anhydride

TABLE 3
Interpretation of the ¹H-NMR-spectra of
3-sulfopropionic anhydride CDCl₃

| | shift (δ | | |
|---|---|---|---|
| Proton number | ppm) | Interpretation | Group |
| 1, 2, 3, 4 | 2.45-2.85 | m, methylene | —O₃S—CH2—CH2— |
| | | protons | COO— |

TABLE 4
Interpretation of the decoupled ¹³C-NMR-
spectra of 3-sulfopropionic acid CDCl₃

| Carbon | shift (δ | | |
|---|---|---|---|
| number | ppm) | Interpretation | Group |
| 1 | 47 | Alkyl carbon | O₃S—CH2—CH2— |
| | | | COOH |
| 2 | 31 | Alkyl carbon | O₃S—CH₂—CH2— |
| | | | COOH |
| 3 | 174 | Carbonyl carbon | O₃S—CH₂—CH2— |
| | | | COOH |

Both spectra were compared and confirmed with reference spectra.

NHS-ester from 3-propionic anhydride:

TABLE 5
Intepretation of the ¹H-NMR-spectra in CDCl₃

| Proton | Shift (δ | | |
|---|---|---|---|
| number | ppm) | Interpretation | Group |
| 1, 2 | 3.20 | m, methylene | O₃S—CH2—CH2—COO— |
| | | protons | |
| 3, 4 | 3.08 | m, methylene | O₃S—CH2—CH2—COO— |
| | | protons | |
| 5, 6, 7, 8 | 2.80 | s, methylene | —CO—CH2—CH2—CO— |
| | | protons | |
| DIEA | 3.67 | m, methine | (CH3)₂CH—N(C2H5)— |
| (2 protons) | | protons | CH(CH3)₂ |
| DIEA | 3.20 | m, methylene | —N—CH2—CH3 |
| (2 protons) | | protons | |
| DIEA | 1.40 | dd, methyl | ((CH3)₂—CH)₂N—CH2—CH3 |
| (15 protons) | | protons | |

Typical inpurities in the crude product are NHS and DIEA. NHS gives a peak at δ82.68(s) and DIEA gives peaks at almost the same ppm as seen above in the table. This makes the DIEA impurity harder to spot than NHS but it can be estimated by looking at the integral of the peaks. If there are any solvent left the MeOH gives a peak at δ 3.49(s), EtOAc at δ2.05(s), δ1.26(t) and at δ4.12(q) and finally DCM at δ5.30(s).

### EXAMPLE 6

### Alternative Preparation of 3-sulfopropionic acid N-hydroxysuccinimide ester

Preparation of 3-sulfopropionic acid

A 1L 3-neck flask was fitted with mechanical stirrer, thermometer and N₂ inlet, an addition funnel, and a heating mantle and set up in an efficient fume hood. Acetic acid, 165.4 ml, was added to the vessel as was 165.4 ml of 30% H₂O₂, 1.46 mole. This mixture was stirred and heated to 50 deg. C. At 50 deg. C. dropwise addition of 3-mercaptopropionic acid, 50 gm 0.471 mole, was begun after the mantle was removed. The reaction is exothermic requiring external cooling. Temperature was maintained at 50-55 deg. C. with a dry ice/acetone bath. When the addition was complete (required about 5 minutes) the reaction remained exothermic for about 30 minutes then the temperature started to drop. When the exothermic activity had ceased, the mantle was replaced and used to maintain the temperature at 50 deg. C. for 2 more hours. Periodic testing of the solution using starch iodide paper indicated the continued presence of peroxide. After 2 hours the clear, colorless solution was allowed to cool and was transferred to a flask for flash evaporation. The rotary evaporator bath was set to 50 deg. C. and used a vacuum source of about 5-6 mm Hg. This step was necessary to remove as much acetic acid as possible so as not to interfere with the subsequent extraction with ethyl acetate. When no more acetic acid/water/H₂O₂ could be collected at this temperature and vacuum (about 1-1.5 hr), the sample was removed and weighed about 100-120 gm. This is greater than the 72 gm theoretical weight of the product and represents water that is very difficult to remove using our evaporative techniques. Freeze drying did not work to remove additional water as the material will not stay frozen even at −20 deg. C. Possibly greatly diluting the material would allow the sample to remain frozen but adding the extra water represents an undesirable step. The concentrated solution was dissolved in 500 ml of water and extracted 3 times with 300 ml each time of ethyl acetate. The ethyl acetate extracts tested positive for H₂O₂ decreasing in intensity with each subsequent extraction. The water layer was concentrated to about 100 gm one final time. The product was a viscous oily product that contained a white precipitate. ¹H NMR analysis in D₂O with a trace of acetonitrile (2.06 ppm) added to serve as an internal standard revealed singlets at 3.23 ppm and 2.78 ppm. Note: these peaks can shift depending on concentration. Minor impurities were observed at 3.58, 2.9, and 2.23 ppm. A ¹³C NMR on the same sample revealed peaks at 174.8, 45.5, and 28.4 ppm.

Preparation of β-sulfopropionic anhydride

The entire sample obtained in the reaction described above (˜100 gm) was treated with 652.4 gm, 5.48 mole, of thionyl chloride again using an efficient fume hood. The thionyl chloride was added incrementally since reaction with the residual water can be vigorous. No violent fuming was observed although HCl and SO₂ are evolved which were directed to the rear of the fume hood using tygon tubing attached to the top of the condenser using an adapter. When addition was complete, the mixture was stirred magnetically at reflux for 12 hours. While cooling yet still stirring the β-sulfopropionic anhydride precipitated. The flask was stoppered and placed in the freezer for 2 hours to maximize the amount of precipitate. The solid anhydride was then collected by filtration in a glove bag under N₂ and the filter cake rinsed twice with 50 ml portions of petroleum ether. The use of the glove bag (a dry box would work as well) is very important since the anhydride is extremely water sensitive reacting to give the starting 3-sulfopropionic acid. The solid anhydride was transferred to a stoppered flask inside the glove bag, then removed to a vacuum desicator where it was unstoppered and subject to a 1 mm vacuum over P₂O₅. The dried anhydride weighed 39 gm, a yield of 61%. ¹H NMR analysis in CDCl₃ revealed singlets at 3.8 ppm and 3.45 ppm. A ¹³C NMR on the same sample revealed peaks at 161.9, 48, and 32 ppm. M.p. was 74.6 deg. C. Lit. 76-77 deg. C.

Reproducibility

This entire sequence (both reactions) was repeated using the same scale and techniques. Nearly identical results were observed. The crude material weighed 84 gm. Note: close observation of the mixture following addition of the thionyl chloride revealed that as the water was consumed in the reaction with excess thionyl chloride in 30-45 minutes, a beautiful white solid precipitated that is believed to be the anhydrous 3-sulfopropionic acid. As the stirring at reflux was continued for another hour, this all dissolved and reacted as observed earlier. The final weight of the second sample of β-sulfopropionic anhydride was 40.7 gm. A yield of 63.5%. %. ¹H NMR analysis in CDCl₃ revealed singlets at 3.8 ppm and 3.45 ppm. A ¹³C NMR on the same sample revealed peaks at 161.9, 48, and 32 ppm.

N-Hydroxysuccinimide ester of 3-sulfopropionic acid, diisopropylethylamine salt

A 500 ml 3-neck flask was prepared with magnetic stirring bar, thermometer and N₂ inlet, and addition funnel. 3.9 gm, 0.0338 mole, of N-hydroxysuccinimide was placed into the flask at room temperature. 100 ml of CH₂Cl₂ was added and the mixture stirred as 4.37 gm, 5.9 ml, 0.0338 mole, of diisopropylethylamine were added. Note: the N-hydroxysuccinimide dissolved upon addition of the diisopropylethylamine. 4.6 gm, 0.0338 mole, of β-sulfopropionic anhydride was dissolved in 80 ml of CH₂Cl₂ and added to the stirred solution using the addition funnel. The reaction mixture darkened as the addition progressed. When addition was complete, the mixture was stirred for 3 additional hours at room temperature then transferred to a single neck flask and the solvent removed on the rotary evaporator yielding a light brown solid residue. The residue was dissolved in 50 ml of CH₂Cl₂ and stirred for 1 hour at room temperature with 2 gm of activated charcoal followed by filtration through glass fiber filter paper and a bed of celite. The celite was rinsed once with 25 ml of CH₂Cl₂. The CH₂Cl₂ was removed on the rotary evaporator. The solid residue was dissolved in 20 ml of 50 deg. C. methanol. This solution was poured into 180 ml of ethyl acetate and the solution placed in the freezer overnight. The next morning a tan solid had precipitated that was collected by filtration. The solid was rinsed on the filter paper with about 50 ml of cold (freezer temperature) ethyl acetate. This filtration was performed in a N₂ filled glove bag although the ester may be expected to have far less water sensitivity than the starting anhydride, if any. The dried sample weighed 7.3 gm and represents a yield of 86%. An ¹HNMR in CDCl₃ revealed: 9.175 (1H-bs),3.6 ppm (2H-m), 3.1 ppm (4H-s), 3.0 ppm (2H-m), and 1.35 ppm (15H-m). A ¹³C NMR on the same sample revealed peaks at 173.3, 168.8, 167.4, 53.9, 45.7, 42.2, 27.4, 25.3, 18.3, 17.1, and 11.9 ppm. The sample had a m.p. of 175-176 deg. C. Lit. 176-178 deg. C.

Note: Care should be taken to use a minimum amount of the methanol/ethyl acetate solvent for the recrystallization step. Too much may result in little or no precipitation of product.

### EXAMPLE 7

### Preparation of 2-sulfobenzoic acid N-hydroxysuccinimide ester

The N-hydroxysuccinimide (NHS) ester of 2-sulfo benzoic cyclic anhydride was prepared as DIPEA salt according to scheme 3 and as explained below:

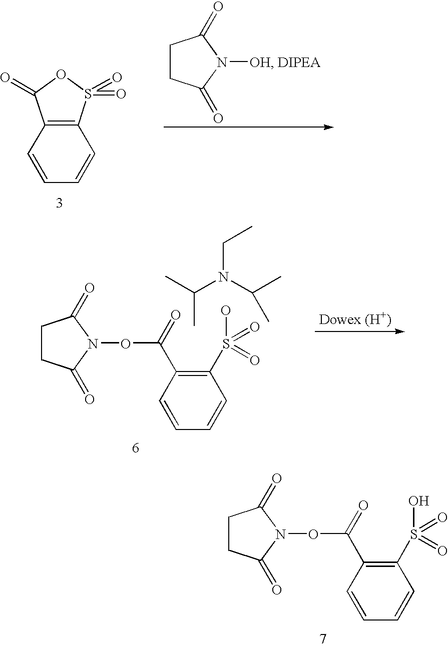

All equipment was dried in an oven and transferred in an exiccator filled with argon prior to use. The reaction was carried out under an argon atmosphere. NHS and 2-sulfo benzoic acid cyclic anhydride were dried under vacuum prior to use. Methylene chloride (1.9 ml) and DIEA (1.019 ml, 5.85 mmol) were added to a round bottle flask containing NHS (673.2, 5.85 mmol). A solution of 2-sulfo benzoic acid cyclic anhydride (1.077 g, 5.85 mmol) in methylene chloride (19 ml) was then added in portions (7×) to the reaction mixture, which was then left at room temperature for 2 h 20 min. The reaction mixture was split in two parts, which were evaporated to give a light yellow highly viscous residue (1.1.11 g and 2.1.24 g, respectively).

Fraction 1 was dissolved in MQ (11.098 ml, 100 mg/ml), filtered and used 3×1 ml in reversed phase preparative HPLC; Column: Supelcosil LC-18, 10 cm×21.2 mm, 2 μ; Flow: 10 ml/min, Method: 0-10 min. isocratic 5% acetonitrile containing 0.1% TFA B in water, 2 min. sample injection, 10-15 min. Gradient 5-12% B in water. The fractions were evaporated and freeze dried to give a white solid/transparent viscous oil (totally 237.7 mg) of not purified product in DIEA salt form, NHS, DIEA and side product. A previous more successful attempt using reversed phase preparative HPLC with the same column and system but another method: 0-6 min. isocratic 5% acetonitrile containing 0.1% TFA B in water, 2 min. sample injection, 6-18 min. Gradient 5-25% B in water, resulted in the product as a DIEA salt with approximately 5% NHS left and some traces from side-product in the aromatic area.

H¹ NMR (D₂O) δ:8.0-8.1 (dd, 1H) 7.9-8.0 (dd, 1H) 7.7-7.8 (m, 2H) 3.6-3.8 (m, 2H) 3.1-3.2 (m, 2H) 3.0 (s, 4H) 1.2-1.3 (m, 15 H) and 2.7 (s, 0.2 H, NHS peak).

Acetone (2.5 ml cold, 0° C., ice-water bath) was added to fraction 2 dropwise to give a white precipitation after 20 min. in room temperature and 25 min. in 4° C. The precipitate was filtered and washed carefully in acetone (24 ml cold, 0° C., ice-water bath) to give the product as a DIEA salt (612.7 mg, 46.3%).

H¹ NMR (D₂O) δ:8.0-8.1 (dd, 1H) 7.9-8.0 (dd, 1H) 7.7-7.8 (m, 2H) 3.6-3.8 (m, 2H) 3.1-3.3 (m, 2H) 3.0 (s, 4H) 1.2-1.3 (m, 15 H).

### EXAMPLE 8

### Synthesis of Another Type of NHS-ester

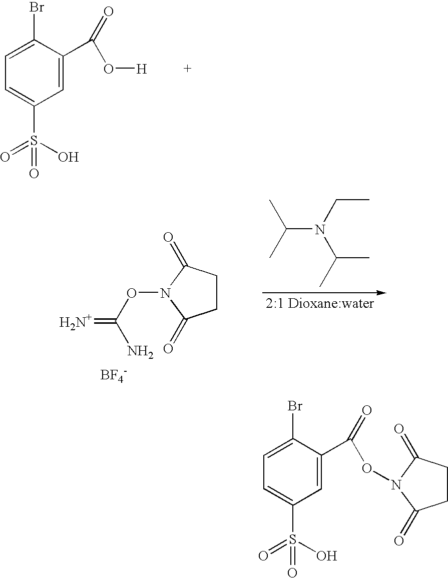

2-bromo-5-sulfobenzoic acid is dissolved in 1 mL dioxane and 0.5 mL water. The diisopropylethylamine, 2 eq., is added. To this well stirred solution is added the O-(N-Succinimidyl)-N,N,N′,N′-tetramethyluronium BF₄ (TSTU), 1.2 eq., as a solid. The reaction is stirred for 30 minutes then concentrated by rotary evaporation followed by drying under high vac. A silica gel column is prepared with 2% water:acetonitrile as the mobile phase. The sample is loaded in 2% water:acetonitrile. The column is started with 2% water:acetonitrile and polarity is progressively increased to 5% water:acetonitrile and finally 80 mL 10% water:acetonitrile. The fractions containing product are identified by TLC in 10% water acetonitrile and confirmed by negative ion MS. This material has approximately 1 equivalent of DIEA by NMR.

### EXAMPLE 9

### Sulfonation of Peptides

Model peptides and tryptic digests of various proteins were dissolved in about 20 μL of base which was prepared by mixing deionized water with diisopropylethylamine (DIEA) in the ratio of 19:1 v:v. Peptide mixtures from in-gel digests were concentrated to a final volume of about 20 μL and 1 μL of DIEA was added to make the solution basic. 5 μL of sulfonic acid active ester reagent at 100 mg/mL is added and the solution vortexed. The pH of each reaction is checked to ensure that it is still basic and adjusted if necessary. The reaction is allowed to proceed for 30 min. at RT. The samples are acidified with 5 μL of 1 N HCl and cleaned up directly using C₁₈ mini-columns (μC₁₈ ZipTip™, Millipore, Bedford Mass.). The sulfonated peptides were eluted from the columns in 4-20 μL of acetonitrile:H₂O (1:1 v:v) containing 0.1% TFA.

### EXAMPLE 10

### Protection of Lys Side Chains by Guanidination and Subsequent Sulfonation of the Tryptic Peptides

Model peptides and tryptic digests of various proteins were dissolved in about 20 μL of base which was prepared by mixing deionized water with diisopropylethylamine (DIEA) in the ratio of 19:1 v:v. Peptide mixtures from in-gel digests were concentrated to a final volume of about 20 μL and 1 μL of DIEA was added to make the solution basic. Two-μL of aqueous 0.5 M O-methylisourea hydrogensulfate was added and the solutions were vortexed. The pH of each solution was checked, and adjusted if necessary, to insure that they were still basic after addition of the reagent. The reactions were then allowed to proceed at room temperature (RT) for varying lengths of time (a few hours to two days). Typically, the room temperature reactions were allowed to proceed overnight. In the morning, 5 μL of sulfonic acid active ester reagent at 100 mg/mL is added and the solution vortexed. The pH of each reaction is checked to ensure that it is still basic and adjusted if necessary. The reaction is allowed to proceed for 30 min. at RT. The samples are acidified with 5 μL of 1 N HCl and cleaned up directly using C₁₈ mini-columns (μC₁₈ ZipTip™, Millipore, Bedford Mass.). The guanidinated-sulfonated peptides were eluted from the columns in 4-20 μL of acetonitrile:H₂O (1:1 v:v) containing 0.1% TFA.

### EXAMPLE 11

### Experimental Description of the Instrument Used (FIG. 3)

Derivatized peptides were analyzed on an Applied Biosystems (Framingham, Mass. 01701) Voyager DE-STR time-of-flight mass spectrometer equipped with a N₂ laser (337 nm, 3 nsec pulse width, 20 Hz repetition rate). All mass spectra were acquired in the reflectron mode with delayed extraction. External mass calibration was performed with low-mass peptide standards, and mass measurement accuracy was typically ±0.2 Da. PSD fragment ion spectra were obtained after isolation of the appropriate derivatized precursor ions using timed ion selection. Fragment ions were refocused onto the final detector by stepping the voltage applied to the reflectron in the following ratios: 1.0000 (precursor ion segment), 0.9126, 0.6049, 0.4125, 0.2738, 0.1975 and 0.1273 (fragment ion segments). The individual segments were stitched together using software developed by Applied Biosystems. All precursor ion segments were acquired at low laser power (variable attenuator=1800) for <256 laser pulses to avoid detector saturation. The laser power was increased (variable attenuator=2100) for the remaining segments of the PSD acquisitions. The PSD data were acquired at a digitization rate of 20 MHz; therefore, all fragment ions were measured as chemically averaged and not monoisotopic masses. Mass calibration was done externally with peptide standards. Metastable ion decompositions were measured in all PSD experiments.

The PSD tandem mass spectra were searched in two ways against the NCBI non-redundant protein sequence database (most recent update at the time of the present filing was Mar. 2, 2001). First, uninterpreted PSD spectra were searched with the MS-Tag program from the Protein Prospector suite of search tools developed at UCSF (see P. R. Baker and K. R. Clauser, http://prospector.ucsf.edu). Search inputs included the measured precursor and fragment ion masses. The measured fragment ion masses of guanidinated peptides were decreased by 42 Da, the mass of the added guanidinium group, before searching against either database. The conservative error tolerances typically used were ±0.6 Da for the monoisotopic precursor ion and ±2.0 Da for the chemically averaged fragment ions. Only y-type fragment ions were allowed possibilities. Other types of fragment ions like a, b, (b+H₂O), (b−NH₃) and internal cleavages were not considered because they are not prominent in the PSD spectra following sulfonation. Alternatively, the PSD data were manually interpreted. The derived sequence tags were searched using the MS-Edman program from the Protein Prospector software package. MS-Edman does not require the precursor or fragment ion masses as inputs. It only uses the measured sequence tags. The program considers all combinations of ambiguous residues, like (K, Q and E) or (I, L, N and D), which have similar masses.

### EXAMPLE 12

### Database Description

The sequences of the polypeptide, and peptides thereof, may also be efficiently and accurately determined using software which accepts mass spectral fragmentation data, either uninterpreted y-ion series masses or sequence tags derived from the y-ion masses, as inputs for sequence database searches. Such search software commonly utilized by the skilled artisan include, but are not limited to, “Protein Prospector” (commercially available from the University of California at San Francisco or http://prospector.ucsf.edu) and “Peptide Search” (commercially available from the European Molecular Biology Laboratory at Heidelberg, Germany or http://www.mann.embl-heidelberg.de).

The fragmentation pattern produced by this invention can be searched against a number of sequence databases including, but not limited to, the NCBI non-redundant database (ncbi.nlm.nih.gov/blast/db.nr.z), SWISPROT (ncbi.nlm.gov/repository/SWISS-PROT/sprot33.dat.z), EMBL (FTP://ftp.ebi.ac.uk/pub/databases/peptidesearch/), OWL (ncbi.nlm.nih.gov/repository/owl/FASTA.z),dbEST (ncbi.nlm.nih.gov/repository/dbEST/dbEST.weekly.fasta.mmddyy.z) and Genebank (ncbi.nlm.nih.gov/genebank/genpept.fsa.z). The entire sequence of the polypeptide of interest can often be retrieved from the sequence database by searching the fragmentation data produced from one or more of the relevant peptide derivatives formed using the methods of this invention.

Of course, when using database searching techniques, it is most efficient to limit the searches by specifying that only y-ions or (y-NH3) ions are allowed fragments because y- and (y-NH3) ions are the most prominent species observed in the fragmentation patterns wherein the present methods are utilized. Other fragment ion types like a-, b-, (b+H2O), (b−H2O), (b−NH3) and internal cleavage ions can be disallowed because they are not prominent in the spectra of the peptides derivatized using the methods of the present invention. The derivatives formed with the present invention provide simple fragmentation patterns that often yield greater database search specificity than can be obtained from the spectra of the same peptides without derivatization.

### EXAMPLE 13

### dPSD of NHS-ester Derivatized Peptides

dPSD of NHS-ester Derivatized Tryptic Digest of a Model Protein:

4-vinyl-pyridine alcylated bovine serum albumin (4VP-BSA) (Sigma) was used as model protein for dPSD using NHS-esters.

Acylation with vinyl-pyridine: The lyophilised protein (2.4 mg) was dissolved in 800 μl of a buffer solution consisting of 8M urea, 50 mM Tris-HCl pH 8.0 and 50 mM DTT and incubated at 30° C. for 30 min. 10μl 4-vinyl pyridine was added (to prevent formation of disulfide bonds) and the sample was incubated for another 1 h at 30° C. The sample was desalted using a NAP-10 column (Amersham Pharmacia Biotech), equilibrated with 100 mM NH₄HCO₂, pH8.8 and eluted in 1.2 ml.

The sample was digested with trypsin (Promega), 1 μg trypsin/100 μg protein, for 6 h at 30° C. and the reaction was stopped by the addition of TFA to a final concentration of 1%. The digest was diluted in 50% AcN:0.5% TFA to a final concentration of 100 ng/μl (1.5 pmol/μl).

N-terminal derivatization with NHS-ester of 3-Sulfopropionic acid anhydride: Tryptic digest of 4VP-BSA (3pmole) were dried on a speed vac and reconstituted in 10 μl of deionized H₂O:diisopropylethylamine (19:1,v:v). The NHS-ester was dissolved in deionized H₂O (10 mg NHS-ester/100 μl H₂O) and 5 μl were added to each sample. The reaction mixture was vortexed and left for 15 minutes at room temperature to react.

The samples were acidified by adding 1 μl 10% TFA and purified through μC₁₈ Zip-Tip™ (Millipore) according the instructions of the manufacturer. The sample was eluted directly on the MALDI-target with a saturated solution of alpha-cyano-4-hydroxycinnamic acid in 50% AcN:0.1% TFA and analyzed in reflectron positive mode and PSD mode positive mode using the Ettan™ MALDI-ToF.

dPSD of NHS-ester Derivatized Tryptic Digests of Proteins from E-coli

Preparation of low speed supernatant of Escherichia coli—Escherichia coli (E-coli), (40 μg stain B, ATCC 11303) was put in 20 ml reducing buffer containing 8M urea/4% chaps, 2% 3-10 pharmalyt, 65 mM DTT. The cells were disrupted by sonication (7×20s with cooling on ice in between). The lysate was centrifuged at 10.000×g for 40 min at 8° C., The low speed supernatant (LSS) was stored in −20° C. until used.

Separation by 2-dimensional (2D) electrophoresis—LSS of E-coli (1 mg) was diluted in IPG rehydration buffer (8M urea/2% CHAPS/2% IPG buffer 4-7/10 mM DTT) and rehydrated into the IPG strips (24 cm, pH 3-10 NL, Amersham Pharmacia Biotech) overnight. 2D-electophoresis was performed following the instructions of the manufacture. After separation by 2D-electrophoresis, the gels were fixed in 40% ethanol (EtOH), 10% acetic acid (HAc) for 1 h, stained with, 0.1% Commassie brilliant blue in 40% EtOH, 10% HAc, for 30 min and destained in 20% EtOH, 5% HAc overnight.

Trpsin digestion: Spots of proteins (1.4 mm in diameter) of medium (˜low pmole) to low intensity (˜high fmole) were picked and transferred to a microtiter plate using the Ettan™ spot picker (Amersham Pharmacia Biotech). The proteins were destained with 100 ul, 50% methanol, 50 mM ammonium bicarbonate (AMBIC), 3×30 minutes, dried in a TuboVap for 15 minutes and digested with 5 ul trypsin for 60 minutes at 37° C. (40 ng/ul 20 mM AMBIC, Promega) using the Ettan™ TA Digester (Amersham Pharmacia Biotech). The peptides were extracted using 35 ul 50% acetonitrile, 0.5% TFA 2× 20 minutes. The extracts were dried at room temperature overnight.

N-terminal derivatization: The samples were reconstituted in 20 μl deionized H₂0. One μl (20%) of each sample was mixed 1:1 with alpha cyano matrix solution and analysed in reflectrone positive mode using the Ettan™ MALDI-ToF. To the remaining 19 μl of each sample, 1 μl DIEA and 5 μl sulfopropionic NHS-ester solution, 10 mg/100 μl were added. The samples were thoroughly mixed by pipeting and left to react for 15 minutes at room temperature. TFA (1 μl, 10%) was added to each sample and purified through μC₁₈ ZipTip™ (Millipore). The samples were eluted directly on the MALDI-target with a saturated solution of alpha-cyano-4-hydroxycinnamic acid in 50% AcN:0.1% TFA and analyzed in reflector positive mode and PSD positive mode using the Ettan™ MALDI-ToF.

Automated dPSD using NHS-esters

The current chemistry is well suited for automation. Using Ettan™ digester and Ettan™ spotter the sample handling and reaction mixtures can be automatically processed. Experimentally, the model peptides or peptide mixtures placed in individual wells of a microtiterplate are reconstituted in 100 ul water (quality of 18 MΩ or better). At this point the liquid handler can split the sample into two reactions. One, containing 5 ul, for direct analysis in the MS, and the other for chemical modification. The material designated for chemical modification is dried at room temperature for one hour. The handler (e.g. a Gilson 215 multiprobe) then reconstitutes the dried material by addition of 10 ul of the reactive derivatisation reagent in a buffer containing DIEA (Diisopropylethylamine). The reactants are mixed by repeated aspiration. The chemical modification step is allowed to proceed for approximately 15 minutes at room temperature. The samples are finally worked up in the same fashion as previously described, and analysed in the MS.

Results

Quantitative N-terminal derivatization of tryptic peptides of 4VP-BSA was obtained with NHS-ester of 3-sulfopropionic acid anhydride in aqueous solution. FIGS. 4 and 5 show the reflectron spectra of non-derivatized and derivatized 4VP-BSA respectively. The peptides I-III were used for dPSD analyses, (FIG. 6-8). The fragmentation spectra showed exclusively y-ions. The fragmentation data from each of the three peptides could be used for unambiguous identification against the NCBInr protein sequence database (PepFrag, www.proteometric.com).

Two gel plugs, containing proteins of E-coli from a commassie stained 2D-gel were identified with dPSD using NHS-ester. The proteins were digested with trypsin, extracted from the gel plug and derivatized as described. FIGS. 9 and 10 show the reflectrone spectra of non-derivatized and derivatized sample from one of the gel plugs. The peptide marked with a circle was quantitatively derivatized and used for PSD analysis (FIG. 11). The masses of the fragment ions (y-ions) were used for protein identification in PepFrag. The suggested candidate from PepFrag agreed with the candidate obtained by searching the tryptic map in ProFound (proteometrics.com). Reflectron spectra of non-derivatized and NHS-ester derivatized sample from the second gel plug are shown in FIGS. 12 and 13. The peptide, m/z 1569 was quantitatively derivatized (m/z 1705) and used for PSD analyses (FIG. 14). The y-ions obtained were used for protein identification in PepFrag, showing the same candidate as obtained with peptide masses in ProFound.

It is apparent that many modifications and variations of the invention as hereinabove set forth may be made without departing from the spirit and scope thereof. The specific embodiments described are given by way of example only, and the invention is limited only by the terms of the appended claims.

1. A method of identifying a polypeptide, which method comprises the steps of
(a) derivatizating the N-terminus of the polypeptide, or the N-termini of one or more peptides of the polypeptide, with at least one acidic reagent containing a sulfonyl or sulfonic acid moiety coupled to an activated ester moiety to provide one or more peptide derivatives, which reagent exhibits a half-life in aqueous solution of not less than 10 minutes at room temperature, to prepare one or more derivatives;
(b) analyzing at least one said derivative using a mass spectrometric technique to provide a fragmentation pattern; and
(c) interpreting the fragmentation pattern obtained to identify the polypeptide,
wherein the peptide or polypeptide is immobilized to a solid support at least during step (a).

2. The method according to claim 1, wherein said solid support is comprised of a silica-based medium derivatized with C₁₈.

3. The method according to claim 1 or 2, wherein step (a) is performed in a solution buffered to a pH within the range of 8-12, such as 9-10.

4. The method according to any one of the preceding claims, wherein the amount of unwanted ester side-products after step (a) is reduced or eliminated by adding one or more nucleophilic reagents to the derivatized polypeptide, followed by a washing step.

5. The method according to claim 4, wherein the amount of unwanted ester side-products after step (a) is reduced or eliminated by adding hydroxylamine hydrochloride to the derivatized polypeptide, followed by a washing step.

6. The method according to any one of the preceding claims, wherein the acidic reagent has a pKa of less than about 2 when coupled to the polypeptide.

7. The method according to any one of the preceding claims, wherein the mass spectrometric technique used in step (b) is matrix-assisted laser desorption ionization (MALDI) mass spectrometry.

8. The method according to any one of the preceding claims, wherein the mass spectrometric technique used in step (b) is electrospray ionization (ESI).

9. The method according to any one of the preceding claims, wherein in step (c), the fragmentation pattern is interpreted using a software program or database.

10. The method according to any one of the preceding claims, wherein all the steps are conducted as part of an automated or semi-automated procedure.

11. The method according to any one of the preceding claims, wherein the activated acid moiety is an N-hydroxysuccinimide (NHS) ester.

12. The method according to any one of the preceding claims, wherein the reagent comprises a 3-sulfopropionic acid N-hydroxysuccinimide ester.

13. The method according to any one of claims 1-12, wherein the reagent comprises a 2-sulfobenzoic acid N-hydroxysuccinimide ester.

14. The method according to any one of the preceding claims, wherein the polypeptide has been obtained by enzymatic digestion.

15. The method according to claim 14, wherein the enzyme is trypsin.

16. The method according to any one of the preceding claims, wherein step (a) is performed during centrifugation of polypeptide and reagent.

17. The method according to any one of the preceding claims, which further comprises a step of protecting lysine residues prior to the sulfonation step.

18. The method according to any one of the preceding claims, which comprises a step of protecting lysine residues before the sulfonation according to step (a), which protection is also performed on peptide(s) and/or polypeptide(s) immobilized to a solid support.

19. A method of protecting lysine residues of peptides and/or polypeptides during a reaction for sulfonation thereof, wherein the peptides are immobilized to a solid support and guanidinated as immobilized before said reaction.

20. A reagent comprising a sulfonyl or sulfonic acid moiety coupled to an activated ester moiety for use in the method of any one of claims 1-19.

21. A reagent suitable for use in peptide derivatization methods wherein the polypeptide is immobilized to a solid support, which reagent is selected from the group consisting of 3-sulfopropionic acid N-hydroxysuccinimide ester and 2-sulfobenzoic acid N-hydroxysuccinimide ester.

22. A kit for identifying a polypeptide by a mass spectrometric technique, which kit comprises at least one reagent in the form of a sulfonyl or sulfonic acid moiety coupled to an activated acid moiety in a container, which reagent exhibits a half-life in aqueous solution of not less than 10 minutes, preferably not less than about 20 minutes and most preferably not less than about 30 minutes at RT.

23. The kit according to claim 22, which further comprises a buffer at a pH of about 8-12, such as 9-10, in a compartment separate from the reagent.

24. The kit according to claim 22 or 23, which also comprises hydroxylamine hydrochloride in a separate compartment.

25. The kit according to any one of claims 22-24, wherein the reagent has a pKa of less than about 2 when coupled to the polypeptide.

26. The kit according to any one of claims 22-25, wherein the mass spectrometric technique is matrix-assisted laser desorption ionization (MALDI) mass spectrometry.

27. The kit according to any one of claims 22-26, wherein the mass spectrometric technique is electrospray ionization (ESI).

28. The kit according to any one of claims 22-27, wherein the activated acid moiety is an N-hydroxysuccinimide (NHS) ester.

29. The kit according to any one of claims 22-28, wherein the NHS ester is selected from the group consisting of 3-sulfopropionic acid N-hydroxysuccinimide ester and 2-sulfobenzoic acid N-hydroxysuccinimide ester.
